(19) United States
(12) Patent Application Publication
(10) Pub. No.: US 2009/0170067 A1
(43) Pub. Date: Jul. 2, 2009

(54) Simian tropic, recombinant human immunodeficiency-1 viruses

(75) Inventors: Theodora Hatziioannou, New York, NY (US); Paul D. Bieniasz, New York, NY (US)

Correspondence Address: Medlen & Carroll, LLP, Suite 350, 101 Howard Street, San Francisco, CA, 94105, US

(73) Assignee: The Rockefeller University, New York, NY (US)

(21) Appl. No.: 11/977,883

(22) Filed: Oct. 25, 2007

Related U.S. Application Data
(60) Provisional application No. 60/854,066, filed on Oct. 25, 2006.

(51) Int. Cl. C12Q 1/70; C12N 15/00; C12N 7/00; C12N 5/06; A01K 67/027

(52) U.S. Cl. 435/5; 435/320.1; 435/235.1; 435/363; 800/14

(57) ABSTRACT

The present invention relates to a vector for producing recombinant human immunodeficiency virus 1 (HIV-1) that is capable of infecting simian cells and monkeys. The recombinant HIV-1 overcomes blocks to infection mediated by simian cell gene products. Such recombinant viruses are useful for evaluating the effectiveness of antiretroviral therapies and vaccines.

This application claims the benefit of U.S. Provisional Application No. 60/854,066, filed on Oct. 25, 2006, herein incorporated by reference in its entirety.

This invention was made with in part with government support under R01AI64003 awarded by the National Institutes of Health, National Institute of Allergy and Infectious Diseases, and N01-CO-12400 awarded by the National Cancer Institute. As such the government has certain rights in the invention.

### FIELD OF THE INVENTION

The present invention relates to a vector for producing recombinant human immunodeficiency virus 1 (HIV-1) that is capable of infecting simian cells and monkeys. The recombinant HIV-1 overcomes blocks to infection mediated by simian cell gene products. Such recombinant viruses are useful for evaluating the effectiveness of antiretroviral therapies and vaccines.

### BACKGROUND OF THE INVENTION

HIV-1, the predominant cause of AIDS in humans, is unable to replicate in most nonhuman primate species except chimpanzees, where infection is typically apathogenic (1, 2). In the case of rhesus macaques, the inability of HIV-1 to establish productive infection appears to be due, at least in part, to blocks in viral replication that are also evident in vitro. HIV-1 replication in macaque cells fails early in the replication cycle because a post-entry block is imposed by a saturable restriction factor, recently identified as TRIM5α, which targets the incoming HIV-1 capsid (CA) (3-10). TRIM5-mediated restriction can occur rapidly (within minutes) after virus entry into the target cell (11) and generally results in blockade of virus replication before the completion of reverse transcription (4, 7-9). Notably, variation in CA-targeted restriction in primate cells is due primarily to variation in TRIM5α sequence (8, 12-15) and the rhesus macaque variant (rh) of TRIM5α potently inhibits HIV-1 but not SIV_{MAC} infection. Overall, the characteristics of TRIM5α-mediated restriction of HIV-1 are similar to those of Fv1-mediated murine leukemia virus restriction (16, 17). Although Fv1 and TRIM5α can be saturated by large doses of incoming virions during in vitro experiments, restriction by Fv1 in vivo dramatically attenuates viral replication and pathogenesis, suggesting that HIV-1 restriction by TRIM5α should be an important determinant of replication in vivo.

A second block to HIV-1 replication in primates may be imposed by the APOBEC3 family of cytidine deaminases. Human APOBEC3G is the major cellular target of HIV-1 Vif (18), which recruits an ubiquitin ligase complex to induce proteasome-dependent APOBEC3G degradation (19-23). In the absence of Vif, APOBEC3G is packaged into virions and subsequently catalyses the deamination of nascent DNA during reverse transcription (24-27). The consequence of minus-strand cytidine deamination is viral DNA instability and G-to-A hypermutation, which is generally lethal to retroviruses (reviewed in 22). Other family members, particularly APOBEC3F, also exhibit antiretroviral activity and some are targeted by Vif, although they generally appear less potent than APOBEC3G (28-32). Notably, even though HIV-1 Vif can efficiently counteract human APOBEC3G and 3F, it is inactive against rhesus or African green monkey APOBEC3G (33).

Thus more HIV-1-like viruses capable of establishing infections in simian cells and monkeys are needed in the art.

### SUMMARY OF THE INVENTION

The present invention relates to a vector for producing recombinant human immunodeficiency virus 1 (HIV-1) that is capable of infecting simian cells and monkeys. The recombinant HIV-1 overcomes blocks to infection mediated by simian cell gene products. Such recombinant viruses are useful for evaluating the effectiveness of antiretroviral therapies and vaccines.

In one embodiment, a recombinant human immunodeficiency virus 1 is provided that overcomes simian cell capsid targeted restriction, overcomes simian cell APOBEC3 family restriction, or the combination thereof. In another embodiment, simian cell capsid targeted restriction is TRIM restriction. In another embodiment, TRIM restriction is TRIM5α restriction. In another embodiment, the virus is capable of infecting normal simian cells, transformed simian cells, normal human cells, transformed human cells, or monkeys. In a further embodiment, the simian cells are rhesus macaque cells, pigtail macaque cells or African green monkey cells, and the monkeys are rhesus macaques, pigtail macaques or African green monkeys. In another embodiment, capsid restriction is overcome by replacement of the human immunodeficiency virus 1 capsid sequence or a fragment thereof by the simian immunodeficiency virus capsid sequence or a fragment thereof. In another embodiment, residues 1-204 of the human immunodeficiency virus 1 capsid sequence are replaced by residues 1-202 of the simian immunodeficiency virus capsid sequence. In another embodiment, the simian immunodeficiency virus is MAC₂₃₉. In a further embodiment, the simian cell APOBEC3 family is APOBEC3F, APOBEC3G or APOBEC3H. In another embodiment, simian cell APOBEC3G restriction is overcome by replacement of the human immunodeficiency virus 1 Vif protein sequence or a fragment thereof by the simian immunodeficiency virus Vif protein or a fragment thereof, while retaining Vpr expression. In a further embodiment, the 5′ end of the simian immunodeficiency virus Vif protein sequence is immediately 3′ to the human immunodeficiency virus 1 Pol stop codon, and the 5′ end of the human immunodeficiency virus 1 Vpr sequence is immediately 3′ to the simian immunodeficiency virus Vif stop codon. In yet a further embodiment, the simian immunodeficiency virus is MAC₂₃₉. In further embodiment, the virus comprises mutations in Gag selected from E12K, K110I, A208V, P371L, or any combination thereof, or a silent mutation in nucleotides 291, 321, or 477 in gag, or 579, 1248, 2149, 2157 or 3411 inpol, or any combination of any of the foregoing. In another embodiment, the virus has greater than about 80% of the human immunodeficiency virus 1 genome, and in a further embodiment, greater than about 87% of the human immunodeficiency virus 1 genome.

In another embodiment, the nucleotide sequence of the recombinant virus from the BssHII site to the SalI site is SEQ ID NO:1 or SEQ ID NO:2.

In another embodiment, a recombinant human immunodeficiency virus 1 is provided comprising a simian immunodeficiency virus capsid sequence or a fragment thereof, a simian immunodeficiency virus Vif sequence or a fragment thereof, or the combination thereof. The virus is capable of infecting normal simian cells, transformed simian cells, normal human cells, transformed human cells, or monkeys. In another embodiment, the simian cells are rhesus macaque cells, pigtail macaque cells or African green monkey cells, and the monkeys are rhesus macaques, pigtail macaques or African green monkeys. In one embodiment, the human immunodeficiency virus 1 capsid sequence or a fragment thereof is replaced by the simian immunodeficiency virus capsid sequence or a fragment thereof. In another embodiment, a simian immunodeficiency virus capsid sequence is provided wherein residues 1-204 of the human immunodeficiency virus 1 capsid sequence are replaced by residues 1-202 of the simian immunodeficiency virus capsid sequence. In one embodiment, the simian immunodeficiency virus is MAC₂₃₉.

In another embodiment, the human immunodeficiency virus 1 Vif protein sequence or a fragment thereof is replaced by the simian immunodeficiency virus Vif protein or a fragment thereof, while retaining Vpr. In another embodiment, the 5′ end of the simian immunodeficiency virus Vif protein sequence is immediately 3′ to the human immunodeficiency virus 1 Pol stop codon, and the 5′ end of the human immunodeficiency virus 1 Vpr sequence is immediately 3′ to the simian immunodeficiency virus Vif stop codon. In another embodiment, the simian immunodeficiency virus is MAC₂₃₉. In other embodiment, the virus comprises mutations in Gag selected from E12K, K110I, A208V, P371L, or any combination thereof, or a silent mutation in nucleotides 291, 321, or 477 in gag, or 579, 1248, 2149, 2157 or 3411 in pol, or any combination of any of the foregoing. In one embodiment, the virus comprises greater than about 80% of the human immunodeficiency virus 1 genome; in another embodiment, is comprises greater than about 87% of the human immunodeficiency virus 1 genome.

In another embodiment, a method is provided for identifying an agent that prevents, attenuates or eliminates human immunodeficiency virus 1 infection in vitro comprising the steps of (a) exposing simian cells to the recombinant virus described above, (b) exposing the simian cells to an agent, (c) determining an effect of the agent on the infection of the simian celis by the strain, and (d) correlating the effect on infection with the ability of the agent to prevent, attenuate or eliminate human immunodeficiency virus 1 infection in vitro. In one embodiment, the simian cells are T cells. In another embodiment, the simian cells are normal simian cells or transformed simian cells, and the simian cells are rhesus macaque cells, pigtail macaque cells or African green monkey cells. In another embodiment, the effect is viral growth, cell survival, reverse transcriptase activity, protease activity, or any combination thereof. In one embodiment the agent is any antiretroviral agent. In one embodiment, the agent is a reverse transcriptase inhibitor, an integrase inhibitor, a protease inhibitor, or an antibody. In another embodiment, step (b) precedes or is concurrent with step (a). In yet another embodiment, the method is a high throughput screen.

In another embodiment, a method is provided for identifying an agent that prevents, attenuates or eliminates human immunodeficiency virus 1 infection in vivo. In one embodiment, the method comprises the steps of (a) exposing a monkey to the recombinant human immunodeficiency 1 virus as described herein; (b) exposing the monkey to an agent, (c) determining an effect of the agent on the infection of the monkey by the virus, and (d) correlating the effect on infection with the ability of the agent to prevent, attenuate or eliminate human immunodeficiency virus 1 infection in vivo. In one embodiment, the monkey is a rhesus macaque, pigtail macaque or African green monkey. In another embodiment, the effect is viral growth, symptoms of AIDS, reverse transcriptase activity, protease activity, viral load, immunologic response, or any combination thereof. In one embodiment the agent is any antiretroviral agent. In another embodiment, the agent is a reverse transcriptase inhibitor, an integrase inhibitor, a protease inhibitor, an antibody or a vaccine. In yet another embodiment, step (b) precedes or is concurrent with step (a).

In another embodiment, a cell or monkey infected with the recombinant human immunodeficiency virus 1 described herein is provided.

Also provided by the present invention are proviral vectors comprising: i) nucleotides encoding a SIVmac capsid protein or fragment thereof in place of nucleotides encoding a HIV-1 capsid protein or fragment thereof; ii) nucleotides encoding a SIVmac Vif protein in place of nucleotides encoding a HIV-1 Vif protein; and iii) nucleotides comprising a sufficient number of nucleotides of an human immunodeficiency virus 1 (HIV-1) genome to provide a chimeric immunodeficiency virus genome suitable for use in producing a chimeric immunodeficiency virus upon introduction into permissive human cells or permissive nonhuman primate cells. In some embodiments, the proviral vector comprises nucleotides encoding an HIV-1 Vpr protein, nucleotides encoding a SIVmac Vpx protein (proviral vector=stHIV(SVpx)). In further embodiments, the proviral vector comprises nucleotides encoding both a SIVmac Vpx protein and a SIVmac Vpr protein in place of nucleotides encoding an HIV-1 Vpr protein (proviral vector=stHIV(SVpx/Vpr)). In further embodiments, the proviral vector comprises nucleotides encoding a SIVmac Nef protein in place of nucleotides encoding an HIV-1 Nef protein (proviral vector=stHIV(SVNef)). In further embodiments, the proviral vector comprises nucleotides encoding a SIVmac Vpx protein, a SIVmac Vpr protein, a SIVmac Tat protein, a SIVmac Rev protein, a SIVmac Env protein, and a SIVmac Nef protein, in place of nucleotides encoding an HIV-1 Vpr protein, an HIV-1 Tat protein, an HIV-1 Rev protein, an HIV-1 Env protein, and an HIV-1 Nef protein (proviral vector=stHIV(SVpx-Nef)). In still further embodiments, the proviral vector comprises nucleotides encoding a green fluorescent protein (GFP or enhanced GFP) in place of nucleotides encoding an HIV-1 Nef protein.

In addition, the present invention provides chimeric immunodeficiency viruses produced upon introduction of a proviral vector described herein, into permissive human cells or permissive nonhuman primate cells. In some preferred embodiments, the permissive nonhuman primate cells are selected from but not limited to rhesus macaque cells, pigtail macaque cells, cynomolgus macaque cells, and African green monkey cells. In further embodiments, the permissive nonhuman primate cells are within a monkey selected from but not limited to rhesus macaques, pigtail macaques, cynomolgus macaques, and African green monkeys.

Moreover, the present invention provides methods for identifying a test agent that modulates human immunodeficiency virus 1 infection in vitro comprising the steps of: a) exposing nonhuman primate cells to either a proviral vector described herein, or to a chimeric immunodeficiency produced upon introduction of the proviral vector into permissive human cells or permissive nonhuman primate cells, to yield infected cells; b) culturing the infected cells in the presence or absence of a test agent; and c) measuring a correlate of infection of the cultured infected cells, whereby a difference in the correlate in the presence of the test agent as compared to in the absence of the test agent, indicates that the agent modulates human immunodeficiency virus 1 infection in vitro. In some preferred embodiments, the nonhuman primate cells are selected from but not limited to rhesus macaque cells, pigtail macaque cells, cynomolgus macaque cells, and African green monkey cells. In further embodiments, the nonhuman primate cells are selected from the group consisting of PBMC, immortalized T cell lines, and immortalized monocyte cell lines. In some embodiments, the correlate of infection is selected from but not limited to viral growth, cell survival, reverse transcriptase activity, integrase activity, and protease activity. In some embodiments, step (b) precedes or is concurrent with step (a).

Also provided by the present invention are methods for identifying a test agent that modulates human immunodeficiency virus 1 infection in vivo comprising the steps of: a) exposing a monkey to either a proviral vector describe herein, or to a chimeric immunodeficiency virus produced upon introduction of the proviral vector into permissive human cells or permissive nonhuman primate cells, to yield an infected monkey; b) treating the monkey with a test agent; and c) measuring a correlate of infection of the treated infected monkey, whereby a difference in the correlate in the treated infected monkey as compared to an untreated infected monkey, indicates that the agent modulates human immunodeficiency virus 1 infection in vivo. In some preferred embodiments, the monkey is selected from but not limited to rhesus macaques, pigtail macaques, cynomolgus macaques, and African green monkeys. In some embodiments, the correlate of infection is selected from but not limited to viral growth, CD4-positive cell depletion, AIDS symptoms, reverse transcriptase activity, integrase activity, protease activity, viral load, and immune response. In some preferred embodiments, the test agent is selected from the group consisting of a reverse transcriptase inhibitor, an integrase inhibitor, a protease inhibitor, an antibody and a vaccine. In some embodiments, step (b) precedes or is concurrent with step (a).

In addition, the present invention provides nonhuman primate cells infected with a chimeric immunodeficiency virus described herein. In still further embodiments, the present invention provides monkeys infected with a chimeric immunodeficiency virus described herein.

In another embodiment, a recombinant human viral vector is provided that overcomes simian cell capsid targeted restriction. In another embodiment, simian cell capsid targeted restriction comprises TRIM restriction. In another embodiment, TRIM restriction comprises TRIM5α restriction. In a further embodiment the viral vector comprises a therapeutic gene. In another embodiment, the viral vector is capable of delivering the therapeutic gene into normal simian cells, transformed simian cells, normal human cells, transformed human cells, humans or monkeys. In a further embodiment, the simian cells are rhesus macaque cells, pigtail macaque cells or African green monkey cells, and the monkeys are rhesus macaques, pigtail macaques or African green monkeys. In another embodiment, simian cell TRIM5α restriction is overcome by the viral vector comprising a capsid sequence of simian immunodeficiency virus or a fragment thereof. In another embodiment, residues 1-202 of the simian immunodeficiency virus capsid sequence replace residues 1-204 of the human immunodeficiency virus 1 capsid sequence. In another embodiment, the simian immunodeficiency virus is MAC₂₃₉. In further embodiment, the capsid sequence comprises mutations in Gag selected from E12K, K110I, A208V, P371L, or any combination thereof, or a silent mutation in nucleotides 291, 321, or 477 in gag, or 579, 1248, 2149, 2157 or 3411 in pol, or any combination of any of the foregoing. In another embodiment, the virus comprises at least one mutation in Gag: K101I, A208V, or P371L.

In another embodiment, the virus comprises mutations in Gag: K110I, A208V, and P371L. In a further embodiment, the therapeutic gene is an antisense oligonucleotide, a replacement gene, a tumor suppressor gene, a gene encoding an inducer of apoptosis, a gene encoding an enzyme, a gene encoding a hormone, a gene encoding an interleukin, or a gene encoding a cytokine. In other embodiments, the therapeutic gene can be a gene for a marker to test the efficiency of gene delivery or expression, such as but no limited to the green fluorescent protein gene.

In yet another embodiment, a recombinant HIV-1 based viral vector is provided comprising a simian immunodeficiency virus capsid sequence or a fragment thereof. In another embodiment the viral vector comprises a therapeutic gene. In one embodiment, a recombinant viral vector comprising a therapeutic gene is provided that overcomes simian cell TRIM restriction. In one embodiment TRIM restriction is TRIM5α restriction. In another embodiment, the viral vector is capable of delivering the therapeutic gene into normal simian cells, transformed simian cells, normal human cells, transformed human cells, humans or monkeys. In a further embodiment, the simian cells are rhesus macaque cells, pigtail macaque cells or African green monkey cells, and the monkeys are rhesus macaques, pigtail macaques or African green monkeys. In another embodiment, simian cell TRIM5α restriction is overcome by the viral vector comprising the simian immunodeficiency virus capsid sequence or fragment thereof. In another embodiment, residues 1-202 of the simian immunodeficiency virus capsid sequence replace residues 1-204 of the human immunodeficiency virus 1 capsid sequence. In another embodiment, the simian immunodeficiency virus is MAC₂₃₉. In further embodiment, the capsid sequence comprises mutations in Gag selected from E12K, K110I, A208V, P371L, or any combination thereof, or a silent mutation in nucleotides 291, 321, or 477 in gag, or 579, 1248, 2149, 2157 or 3411 in pol, or any combination of any of the foregoing. In another embodiment, the virus comprises at least one mutation in Gag: K110I, A208V, or P371L. In another embodiment, the virus comprises mutations in Gag: K110I, A208V, and P371L. In a further embodiment, the therapeutic gene is an antisense oligonucleotide, a replacement gene, a tumor suppressor gene, a gene encoding an inducer of apoptosis, a gene encoding an enzyme, a gene encoding a hormone, a gene encoding an interleukin, or a gene encoding a cytokine. In other embodiments, the therapeutic gene can be a gene for a marker to test the efficiency of gene delivery or expression, such as but no limited to the green fluorescent protein gene.

In another embodiment, a method for evaluating the effectiveness of a therapeutic gene viral vector in monkeys, simian cells, humans or human cells is provided, comprising the steps of (a) exposing monkeys, simian cells, humans or human cells to the viral vector comprising a therapeutic gene described above, (b) determining the level of expression of the therapeutic gene therein or the therapeutic effect thereon, and (c) correlating the expression or effect with the ability of the recombinant viral vector to deliver the therapeutic gene. In one embodiment, the simian cells or human cells are T cells. In another embodiment, the simian cells are normal simian cells or transformed simian cells, and the simian cells are rhesus macaque cells, pigtail macaque cells or African green monkey cells. In another embodiment the human cells are normal human cells or transformed human cells. In another embodiment, the monkey is a rhesus macaque, a pigtail macaque or an African green monkey. Non-limiting examples of therapeutic genes and genes expressing markers are as mentioned above.

### BRIEF DESCRIPTIONS OF THE DRAWINGS

FIG. 1 shows a schematic representation of the organization of the HIV-1 genome and nomenclature of the derivatives used herein. White boxes represent HIV-1 open reading frames, shaded boxes represent SIV_{MAC239} counterparts. Env genes from two HIV-1 strains (HxB or KB9) were used, and some constructs encoded GFP in place of Nef. Restriction enzyme sites used for generating the various recombinant proviruses are indicated.

FIG. 2 shows the capsid expression by and replication of viruses bearing SIV_{MAC} CA. A. Western blot (α-p24CA) analysis of 293T cell lysates (cells) and extracellular virions harvested by ultracentrifugation (virus) after transfection with HIV/HxB (lane 1), HIV(SCA)/HxB (lane 2) or B3HIV(SCA)/HxB (lane 3). The single-cycle infectivity of each virus as measured by enumeration of GFP-positive cells after titration on GHOST cells (infectious units (i.u.) per ng of reverse transcriptase (RT)) is indicated. B. Replication in CEMx174 cells following inoculation with 1 ng (RT) of the indicated viruses. Supernatant samples were analyzed by RT assays every 2-3 days until gross cytopathic effects and cell death were evident.

FIG. 3 depicts the characterization of HIV/KB9/GFP proviral clones expressing SIV_{MAC} CA and/or SIV_{MAC} Vif. A. Western blot (α-p24CA) analysis of expression and particle production by 293T cells transfected with HUV/KB9/GFP (lane 1), HIV(SVif)KB9/GFP (lane 2), B3HIV(SCA)/KB9/GFP (lane 3) and B3HIV(SCA, SVif)/KB9/GFP (lane 4) proviral plasmids. The single-cycle infectivity of each virus, as measured on GHOST cells, in infectious units (i.u.) per ng of RT is indicated. B. Viruses encoding SIV_{MAC}CA are resistant to rhTRIM5α. Human CEMx174 cells, CEMx174 engineered to express rhTRIM5α or rhesus macaque 221 cells were inoculated with HIV/KB9/GFP or B3HIV(SCA)/KB9/GFP expressing HIV-1 or SIV_{MAC}Vif as indicated. C. HIV-1 expressing SIV_{MAC}Vif resists inhibition by rhAPOBEC3G. CEMx174 cells were infected with HIV/KB9/GFP-based virus stocks generated in 293T cells in the presence or absence of rhAPOBEC3G. The left panel shows viruses derived from HIV/KB9/GFP, expressing HIV-1 or SIV_{MAC}Vif proteins as indicated, while the right panel shows viruses derived from B3HIV(SCA)/KB9/GFP expressing HIV-1 or SIV_{MAC}Vif proteins, as indicated. D. Replication of HIV/KB9/GFP-derived viruses in CEMx174 cells. Cells were inoculated with 1 ng of RT (approximate multiplicity of infection=0.02) of HIV-1 Vif-expressing (left panel) or SIV_{MAC}Vif-expressing (right panel) versions of HIV/KB9/GFP or B3HIV(SCA)/KB9/GFP. E. Same as D, except that rhesus macaque 221 cells were inoculated with 10 ng (RT) of the indicated viruses. Supernatant samples were analyzed by RT assays.

FIG. 4 shows stHIV/KB9 replication in human and rhesus monkey T-cell lines. A. Human CEMx174 cells were inoculated with 1 ng of RT of HIV/KB9, SIVMAC239, or stHIV/KB9. Cells were cultivated until gross cytopathic effects became evident and supernatant samples were analyzed by RT assay. B. Same as A except that rhesus macaque 221 cells were inoculated with 1 ng of RT of each virus.

FIG. 5 shows that stHIV retains sensitivity of HIV-1-specific antiretroviral drugs. A, B. TZM-bl indicator cells were inoculated with the indicated virus stocks in the presence of increasing concentrations of AZT (A) or nevirapine (B). The level of infection, determined by luciferase assay, is plotted as a function of drug concentration, as a percentage of the uninhibited level. C. Each virus was generated by infected HuT-CCR5 cell in the presence of increasing concentrations of amprenavir. Infectious virus generation, determined by inoculation of TZM-bl indicator cells, is plotted as a function of amprenavir concentration, as a percentage of that generated in its absence.

FIG. 6 shows the replication of stHIV/KB9 in primary rhesus macaque T-cells. A. CD4+ enriched T-cells from rhesus macaques were inoculated with 0.5 ml of stocks of stHIV/KB9 (21 ng of p27), SIVMAC239 (136 ng of p27) or HIV-1NL4-3 (1550 ng of p24), as indicated. Supernatant samples were assayed by ELISA for p24 (for HIV-1) or p27 (for stHIV and SIV_{MAC}). B. PBMC from macaque donors were inoculated with 1 ng of RT of HIV/KB9, SIVMAC239, or stHIV/KB9. Supernatant samples were analyzed by RT assay.

FIG. 7 shows maps of the pV1 and pCRV1 Gagpol constructs.

FIG. 8 shows a schematic representation of HIV-1/SIV chimeras based on stHIV and containing one or more of SIV Nef, SIV Vpx, and SIV Vpr, or the 3′ end of the SIV genome. White boxes represent HIV-1-derived sequences while gray boxes represent SIV_{MAC}-derived sequences.

FIG. 9 shows the replication of stHIV/YU2 in rhesus macaque PBMC (rhPBMC). Briefly, rhPBMC were inoculated with equivalent RT levels (1 ng/10⁵ cells) for each indicated virus. Supernatant samples were collected every 2-3 days and analysed by RT assay.

FIG. 10 illustrates the adaptation of stHIV variants with a comparison of the replication of viruses produced in 293T (open squares) and viruses collected from infected 221 cells after 24 passages (closed squares). 221 cells were inoculated with an MOI of 0.01 (measured on GHOST cells) of each virus. Supernatant samples collected thereafter were analyzed by TZM infection assay.

FIG. 11 illustrates that stHIV replicates in monkeys. Two rhesus macaques were intravenously injected with 2 ml of stHIV supernatant with titers of 5×10⁵ i.u./ml as measured on GHOST cells (indicator cell line expressing GFP under the control of the HIV-1 LTR and CD4, CXCR4 and CCR5 receptors). Samples were harvested every 3 days and viral load monitored using a real time PCR assay for SIV CA RNA.

FIG. 12 shows the nucleic acid sequence of stHIV from the BssHII site to the SalI site (SEQ ID NO:1).

FIG. 13 shows the nucleic acid sequence of stHIV lacking adaptive changes (SEQ ID NO:2).

### DEFINITIONS

To facilitate understanding of the invention, a number of terms are defined below.

The term “vaccine” as used herein, refers to a composition that is administered to produce or artificially increase immunity to a particular disease. For example, “vaccine compositions” frequently comprise a preparation of killed or live attenuated microorganisms. Alternatively, subunit vaccines frequently comprise a preparation of isolated nucleic acids or proteins corresponding to the genes or gene products of a microorganism of interest.

The term “primate immunodeficiency virus” refers to any lentivirus or to any member of the Lentivirus family, which is capable of causing immune suppression in an infected human or nonhuman primate. In some embodiments, the term “immunodeficiency virus” refers to the retrovirus known as the human immunodeficiency virus (HIV), which is responsible for the fatal illness termed the acquired immunodeficiency syndrome (AIDS). Two kinds of HIV have been identified: HIV-1 is the more virulent, pandemic virus, and HIV-2 is the closely related virus largely confined to West Africa.

As used herein, the term “genome” refers to the total set of genes carried by an organism. In preferred embodiments, the term “genome” refers to the complete set of genes from an immunodeficiency virus. The term “gene” refers to a specific sequence of nucleotides (e.g., DNA or RNA) that is the functional unit of inheritance controlling the transmission and expression of one or more traits.

The terms “Gag” and “group specific antigen” refer to the immunodeficiency virus polyprotein composed of MA, CA, NC, and p6.

The terms “Pol” and “polymerase” refer to the immunodeficiency virus polyprotein composed of the protease, reverse transcriptase, RNaseH and integrase enzymes.

The terms “Env” and “envelope” refer to the immunodeficiency virus polyprotein (e.g., gp160) composed of surface (e.g., gp120) and transmembrane (e.g., gp41) subunits.

As used herein, the term “regulatory protein” refers to the small immunodeficiency virus proteins involved in modulation of the viral replicative cycle, including Tat, and Rev.

As used herein, the term “accessory protein” refers to the small immunodeficiency virus proteins whose functions have been shown to be dispensable in vitro, including but not limited to Nef, Vpu, Vpr, and Vif.

The term “suitable for” as used herein, refers to a condition or a combination adapted to a specific use or purpose. In some embodiments, “suitable for” refers to conditions for administration of a vaccine to a subject; as such this term encompasses but is not limited to an appropriate vaccine dosage (e.g., less than 10 cc), an appropriate vaccine formulation (e.g., alum adjuvant), and an appropriate vaccine schedule (See, e.g., prime plus boost).

As used herein, the term “immune response” refers to the alteration in the reactivity of an organism's immune system upon exposure to an antigen. The term “immune response” encompasses but is not limited to one or both of the following responses: antibody production (e.g., humoral immunity), and induction of cell-mediated immunity (e.g., cellular immunity including helper T cell and/or cytotoxic T cell responses).

The term “route” as used herein, refers to methods for administration of a virus or prophylactic or therapeutic agent (e.g., enzyme inhibitor or vaccine formulation). In some embodiments, “route” refers to the method of administration of a vaccine including but not limited to intramuscular, intravenous, intraperitoneal, subcutaneous, oral, intranasal, intravaginal, intrarectal, and stomacheal administration methods.

As used herein, the term “physiologically acceptable solution” refers to an isotonic solution such as an aqueous solution comprising for example, saline, phosphate buffered saline, Hanks' solution, or Ringer's solution.

The term “infected” as used herein, refers to a subject in which a pathogen has established itself. In preferred embodiments, the term “infected subject” refers to a subject that is infected with an immunodeficiency virus. In contrast, the term “uninfected” refers to a subject, which has not been contaminated with a pathogen. In preferred embodiments, the term “uninfected subject” refers to a subject that is not infected with an immunodeficiency virus. In the context of the invention, the term “uninfected subject” encompasses subjects, which may be infected with other types of viruses (e.g., CMV, EBV, etc.).

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals, which receive a mock treatment (e.g., empty vector).

As used herein, the term “antibodies reactive with” refers to antibodies, which bind to or react with an antigen of interest. In preferred embodiments of the present invention, the term “antibodies reactive with” is used in reference to antibodies which bind to the immunodeficiency virus of interest, or viral Gag, Pol, Env, regulatory or accessory proteins.

The term “cytotoxic T lymphocytes reactive with” refers to cytotoxic T lymphocytes capable of lysing an MHC (e.g., HLA)-matched cell presenting epitopes derived from an antigen of interest. In preferred embodiments of the present invention, the term “cytotoxic T lymphocytes reactive with” is used in reference to cytotoxic T lymphocytes or CTLs capable of lysing a MHC-matched cell infected by the immunodeficiency virus of interest, or presenting epitopes derived from viral Gag, Pol, Env, regulatory or accessory proteins.

The term “helper T lymphocytes reactive with” refers to helper T lymphocytes capable of secreting lymphokines in response to an MHC (e.g., HLA)-matched cell presenting epitopes derived from an antigen of interest. In preferred embodiments of the present invention, the term “helper T lymphocytes reactive with” is used in reference to helper T lymphocytes or TH cells capable of secreting lymphokines in response to an MHC-matched cell infected by the immunodeficiency virus of interest, or presenting epitopes derived from viral Gag, Pol, Env, regulatory or accessory proteins.

As used herein, the term “induced an immune response” refers to an immune response elicited by a vaccine or a set of vaccines of the present invention.

“Wild-type,” as used herein, refers to a gene or gene product having characteristics of that gene or gene product when isolated from a naturally occurring source. A wild-type gene is that which is most frequently observed in a population and is thus arbitrarily designed the “normal” or “wild-type” form of the gene.

“Mutant,” as used herein, refers to any changes made to a wild-type nucleotide sequence, either naturally or artificially, that produces a translation product that functions with enhanced or decreased efficiency in at least one of a number of ways including, but not limited to, specificity for various interactive molecules, rate of reaction and longevity of the mutant molecule.

As used herein, the term “virulent” refers to markedly pathogenic immunodeficiency viruses (e.g., viruses capable of causing severe disease).

The terms “expression vector,” “expression construct,” “expression cassette” and “plasmid,” as used herein refer to a recombinant nucleic acid molecule containing a desired coding sequence and appropriate nucleic acid sequences necessary for the expression of the operably linked coding sequence in a particular host organism. The sequences may be either double or single-stranded. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The terms also refer to the linkage of amino acid sequences in such a manner so that a functional protein is produced.

As used herein, the term “strain” refers to a group of presumed common ancestry, but with some clear-cut genetic distinctions (e.g., not clones). In preferred embodiments, the term “strain” is used in reference to distinct immunodeficiency virus isolates.

The terms “human immunodeficiency virus type-1” and “HIV-1” refer to the lentivirus that is widely recognized as the aetiologic agent of the acquired immunodeficiency syndrome (AIDS). HIV-1 is characterized by its cytopathic effect and affinity for CD4+-lymphocytes and macrophages. The terms “human immunodeficiency virus type-2” and “HIV-2” refer to a lentivirus related to HIV-1 but carrying different antigenic components and with differing nucleic acid composition. The term “recombinant HIV strain” refers to an HIV virus produced from an immunodeficiency virus genome that has been assembled through the use of molecular biology techniques that are well known in the art.

An exemplary HIV-1 genome is that of HIV-1_{NY5/BRU} of GENBANK Accession No. M19921 (herein incorporated by reference), likewise an exemplary HIV-1 proviral vector is that of pNL4-3 of GENBANK Accession No. AF324493 (herein incorporated by reference).

The terms “simian immunodeficiency virus” and “SIV” refer to lentiviruses related to HIV, which cause acquired immunodeficiency syndrome in nonhuman primates (e.g., monkeys and apes). An exemplary SIV genome is that of SIV_{mac239} of GENBANK Accession No. M33262 (herein incorporated by reference).

The terms “simian human immunodeficiency virus” and “SHIV” refer to various man made chimeric retroviruses having both human and monkey immunodeficiency virus genes. An exemplary SHIV genome is that of SHIV-4 (having an HIV-1_{HXBc2} env gene) of GENBANK Accession No. AF038399 (herein incorporated by reference).

In some embodiments, the nucleotides encoding an SIVmac capsid or portion thereof, comprise a polynucleotide with the nucleotide sequence set forth as SEQ ID NO:1 or SEQ ID NO:2, or a polynucleotide at least 25, 50, 100, 200, 250 or 500 bp in length (preferably at least 1,000 bp, more preferably at least 1,500 bp and most preferably at least 3,000 bp in length) that hybridizes under highly stringent conditions to the nucleotide sequence set forth in SEQ ID NO:1 or SEQ ID NO:2. In some embodiments, the polynucleotide sequence is comprises a portion of the nucleotide sequence set forth as SEQ ID NO:1 or SEQ ID NO:2. In preferred embodiments the portion is greater than or equal to 25 bp in length and less than or equal to 3000 bp in length (e.g., about 25, 50, 100, 200, 250, 500, 1,000, 1,500 or 3,000 bp in length) and hybridizes under highly stringent conditions to the nucleotide sequence set forth in SEQ ID NO:1 or SEQ ID NO:2. In other embodiments the nucleotides encoding an SIVmac capsid or portion thereof, are 80%, 85%, 90%, 95%, 96%, 97%, 98%, 99% or 100% identical to the nucleotide sequence set forth in SEQ ID NO:1 or SEQ ID NO:2. Likewise, in some embodiments the nucleotides encoding a SIVmac protein are 80%, 85%, 90%, 95%, 96%, 97%, 98%, 99% or 100% identical to the nucleotide sequence encoding the protein as set forth in GENBANK Accession No. M33262 (e.g., comprising one to several nucleotide deletions, additions or substitutions). Similarly, in some embodiments, the nucleotides encoding an HIV-1 protein are 80%, 85%, 90%, 95%, 96%, 97%, 98%, 99% or 100% identical to the nucleotide sequence encoding the protein as set forth in GENBANK Accession No. AF324493 (e.g., comprising one to several nucleotide deletions, additions or substitutions).

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “adjuvant” as used herein refers to any compound that when injected together with an antigen, non-specifically enhances the immune response to that antigen. Exemplary adjuvants include but are not limited to incomplete Freunds adjuvant (IFA), aluminum-based adjuvants (e.g., AIOH, AIPO4, etc), and Montanide ISA 720.

The terms “excipient,” “carrier” and “vehicle” as used herein refer to usually inactive accessory substances into which a pharmaceutical substance (e.g., EHEC cells) is suspended. Exemplary carriers include liquid carriers (such as water, saline, culture medium, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins).

As used herein, the term “viral enzyme” refers to viral proteins that catalyze chemical reactions of other substances without being destroyed or altered upon completion of the reactions. The terms “protease” and “Pro” refer to a viral enzyme that catalyses the splitting of interior peptide bonds in a protein. The terms “reverse transcriptase” and “RT” refer to a viral enzyme involved in the synthesis of double stranded DNA molecules from the single stranded RNA templates. The terms “RNase H” and “Ribonuclease H” refer to a viral enzyme that specifically cleaves an RNA base paired to a complementary DNA strand. The terms “integrase” and “IN” refer to a viral enzyme that inserts a viral genome into a host chromosome.

As used herein, the terms “long terminal repeat” and “LTR” refer to homologous nucleic acid sequences, several hundred nucleotides long, found at either end of a proviral DNA, and formed by reverse transcription of retroviral RNA. LTRs are thought to have an essential role in integrating the provirus into the host DNA. In proviruses the upstream LTR acts as a promoter and enhancer and the downstream LTR acts as a polyadenylation site.

The term “adjuvant” refers to a substance added to a vaccine to improve the immune response (e.g., alum). As used herein, the term “molecular adjuvant” refers to proteins that improve the immunogenicity of a vaccine or to the genes, which encode these proteins. The term “molecular adjuvant” encompasses but is not limited to costimulatory molecules, cytokines, chemokines, growth factors, etc.

As used herein the phrases “sufficient number of nucleotides of an human immunodeficiency virus 1 (HIV-1) genome” and “nucleotides of an HIV-1 backbone” when used in the context of a simian tropic HIV-1 (stHIV-1) virion or vector of the present invention refer to a range in the number of nucleotides of HIV-1 needed to complement nucleotides of SIV (and optionally nucleotides of nonviral origin), to yield a chimeric immunodeficiency virus upon introduction into permissive human cells and/or permissive nonhuman primate cells. In some embodiments, the sufficient number of nucleotides of HIV-1 comprise nucleotides encoding one or more of HIV-1 Pol, HIV-1 Vpr, HIV-1 Vpu, HIV-1, HIV-1 Env, HIV-1 Tat, HIV-1 Rev and HIV-1 Nef. In further embodiments, the sufficient number of nucleotides of HIV-1 further comprise one or both long terminal repeats (LTRs). As such the SIV and HIV-1 (or SIV, HIV-1 and nonviral) nucleotides together but not individually comprise a complete immunodeficiency virus genome, in which the aggregate, but not each separate immunodeficiency virus component comprises a complete genome.

### DETAILED DESCRIPTION OF THE INVENTION

Since HIV-1 does not productively infect most non-human primates, the preferred animal models for human HIV-1 infection and AIDS often involve infection of rhesus macaques by SIV_{MAC}-based viruses. However, despite compelling parallels between human and simian AIDS, the fact that SIV_{MAC} and HIV-1 are distinct viruses can limit the usefulness of such models. Primate cells express restriction factors, exemplified by TRIM5α and APOBEC3G, that inhibit retroviral infection; their functional interaction with viral capsid (CA) and Vif proteins, respectively, can restrict retrovirus tropism. To assess whether failure of the HIV-1 CA and Vif proteins to overcome host restrictions accounts for the inability of HIV-1 to replicate in rhesus macaque cells, sequential construction was used by the inventors herein together with adaptation steps to generate an HIV-1-based virus strains, expressing SIV_{MAC}CA and Vif proteins, that are resistant to rhesus macaque TRIM5α- and APOBEC3G-mediated restriction. These viruses replicate robustly in both transformed and primary rhesus macaque T-cells. Derivation of a simian-tropic HIV-1 (stHIV) has profound implications for the development of improved animal models of human AIDS.

Simian or primate as referred to herein comprise species that are not normally infected by human HIV-1, namely, primates other than humans and chimpanzees. By way of non-limiting example, simian or primate species herein include rhesus macaques, pigtail macaques, East African green monkeys, West African green monkeys, sooty mangabeys and mandrills.

Primate cells contain at least two major post-entry restrictors of retroviral replication, namely TRIM5α and APOBEC3G and sensitivity to them is governed by CA and Vif proteins, respectively. HIV-1 has apparently adapted to overcome human variants of these intrinsic inhibitors of retroviral replication, but is sensitive to their counterparts in many nonhuman primates. Previous attempts to derive macaque-tropic HIV-1 strains yielded viruses that replicated only modestly in macaque PBMC (37) and previously derived macaque-tropic SHIV chimeras are primarily SIV_{MAC} in origin (35, 38-42). Therefore, it was unclear whether CA and Vif-based, TRIM5α and APOBEC3G-mediated restriction were solely responsible for the failure of HIV-1 to replicate in rhesus macaque cells. The embodiments described herein indicate that avoiding or overcoming CA and Vif-based restriction are sufficient for zoonotic transmission of primate lentiviruses in general. Importantly, engineering resistance to restriction factors is likely to be a feasible way to develop more authentic animal models of HIV-1 infection. As described herein, the inventors generated minimally chimeric viruses, termed simian-tropic HIV-1 (stHIV), which in one embodiment 88% of the genome is HIV-1-derived, that can replicate robustly in rhesus macaque T-cells. The recombinant viruses are merely exemplary of the viruses that can be generated by the methods described herein. In other embodiments, other simian cell capsid targeted restriction factors are overcome in the recombinant viruses embodied herein. In another embodiment, the restriction factor is a TRIM factor.

While substitution of HIV-1 CA and Vif proteins with SIV_{MAC} counterparts conferred resistance to restriction factors, engineering steps decreased the in vitro replicative capacity of the recombinant virus. Adaptation steps were therefore required to generate stHIV. Nonetheless, a very small number of mutations define the differential properties of the final and the starting constructs. Because the adaptation steps conferred significantly enhanced replication in both human and macaque cells, such adaptations likely were required to accommodate the presence of SIV_{MAC} CA and Vif sequences in the HIV-1 genome, rather than to overcome any additional macaque-specific blocks to HIV-1 replication.

Experimental infection of macaques with SIV_{SM/MAC/MNE} family of viruses has provided key insights into primate lentiviral pathogenesis, and serves as the principal non-human primate models of AIDS. Nonetheless, despite the many attractive features of these models, the inability of HIV-1 to replicate in macaques complicates and limits animal model testing of treatment and prevention strategies. Since SIV_{MAC} and HIV-1 exhibit little immunological cross reactivity, evaluation of AIDS vaccines currently entails initial validation of the protective potential of the vaccine approach in an SIV challenge model, and a separate assessment of the immunogenicity of the corresponding HIV immunogens. While SHIV constructs containing HIV-1 Env sequences (35, 40, 41; U.S. Pat. No. 5,654,195 to Sodroski et al., and U.S. Pat. No. 5,849,994 to Narayan; all herein incorporated by reference in their entirety) can be useful for studies involving Env-mediated protection in macaques, the pathogenesis of infection with most SHIVs encoding HIV-1 Env varies substantially from SIV infection of macaques or HIV-1 infection of humans (43, 44). Moreover, current SHIVs containing HIV-1 Env sequences are not useful for studies of HIV-1 vaccines that protect via Env-independent mechanisms. Clearly, an HIV-1/macaque based model would greatly simplify and streamline the evaluation of the numerous HIV-1 vaccine candidates.

The recombinant viruses described herein and those that can be prepared following the teachings herein provide facile means for evaluating various strategies to block viral entry, replication, infectivity, and other factors that are targets for immunological or pharmaceutical intervention and prevention. Development of HIV-1 vaccines, alternative approaches such as topical and systemic application of inhibitors, as well as pre- and post-exposure prophylaxis, may form the basis of future AIDS prevention strategies amenable for discovery by practice of the embodiments herein. For studies of these approaches, as well as more conventional evaluations of antiretroviral drugs in animal models, it is problematic that at least two major classes of current antiretroviral drugs that potently inhibit HIV-1 replication, namely nonnucleoside RT inhibitors and protease inhibitors, show limited or unpredictable activity against SIV_{MAC}(46, 47). Moreover, for inhibitors that are effective against both HIV-1 and SIV_{MAC}, the mechanisms underlying acquisition of resistance may differ (48). These problems have been only partly overcome by the generation of SHIV chimeras encoding the RT of HIV-1 (42, 49, 50). The viruses embodied herein offer solutions to the aforementioned problems.

A non-human primate model of human HIV-1 infection in which most or all of the challenge virus is actually derived from HIV-1 overcomes many problems and may also provide a more authentic model for studies of HIV-1 pathogenesis. The experiments described herein indicate that the development of such an HIV-1-based macaque model should be feasible. While the current generation of stHIV retains some SIV_{MAC}-derived sequence, it is clearly much more HIV-1-like than any previously constructed chimera that can replicate robustly in rhesus macaque T-cells. Moreover, it retains sensitivity to two distinct classes of HIV-1 specific antiretroviral agents.

Thus, in one embodiment, a recombinant human immunodeficiency virus 1 capable of growing in monkey cells is prepared by replacement of the human immunodeficiency virus 1 capsid sequence or a fragment thereof by the simian immunodeficiency virus capsid sequence or a fragment thereof, and replacement of the human immunodeficiency virus 1 Vif protein sequence or a fragment thereof by the simian immunodeficiency virus Vif protein or a fragment thereof. These modifications of the human immunodeficiency virus 1 genome are achieved while also retaining the Vpr sequence. The teachings herein and the examples below describe exemplary means for creating the recombinant virus, but these examples are non-limiting and the skilled artisan can readily create alternate viruses by following the guidance herein. As noted herein, adaptation of the modified virus may be required to achieve robust replication in the new host cells, and any genomic changes, to amino acid sequence or silent mutations in coding or non-coding regions, are likewise embraced in the embodiments herein.

As mentioned above, research on new prophylactic and therapeutic strategies for human immunodeficiency virus 1 (HIV-1) and AIDS has been stymied by the unavailability of animal models of the human disease and in vitro counterparts. SIV infection in monkeys while useful does not adequately facilitate modeling of the human disease. The embodiments described here provide a predominantly human virus that replicates in monkey cells, thus overcoming the aforementioned deficiencies and offers in vitro and animal models in which laboratory discoveries thereon can be directly translated to human benefit. Thus, in another embodiment, a method is provide for identifying an agent that prevents, attenuates or eliminates human immunodeficiency virus 1 infection in vitro comprising the steps of (a) exposing simian cells to the recombinant virus described above, (b) exposing the simian cells to an agent, (c) determining an effect of the agent on the infection of the simian cells by the strain, and (d) correlating the effect on infection with the ability of the agent to prevent, attenuate or eliminate human immunodeficiency virus 1 infection in vitro. The simian cells can be T cells, normal simian cells or transformed simian cells, and the simian cells can be rhesus macaque cells, pigtail macaque cells or African green monkey cells. The effect measured on the infection can be viral growth, cell survival, reverse transcriptase activity, protease activity, or any combination thereof. Such assays are well known to those skilled in the art. The agent can be any antiretroviral drug, such as but not limited to a reverse transcriptase inhibitor, an integrase inhibitor, a protease inhibitor, or an antibody, or may represent an entirely new class of compound or method of therapy. The method also facilitates evaluation of complementary and alternative agents. In another embodiment, step (b) precedes or is concurrent with step (a). In yet another embodiment, the method is a high throughput screen.

In another embodiment, a method is provided for identifying an agent that prevents, attenuates or eliminates human immunodeficiency virus 1 infection in vivo. In one embodiment, the method comprises the steps of (a) exposing a monkey to the recombinant human immunodeficiency 1 virus as described herein; (b) exposing the monkey to an agent, (c) determining an effect of the agent on the infection of the monkey by the virus, and (d) correlating the effect on infection with the ability of the agent to prevent, attenuate or eliminate human immunodeficiency virus 1 infection in vivo. The monkey can be a rhesus macaque, pigtail macaque or African green monkey. The effect measured in the method can be viral growth, appearance or progression of the symptoms of AIDS, reverse transcriptase activity, protease activity, viral load, immunologic response, or any combination thereof. The agent tested can be any antiretroviral drug, such as but not limited to a reverse transcriptase inhibitor, an integrase inhibitor, a protease inhibitor, an antibody or a vaccine, or an entirely new class of compound or even method of treatment such as ex vivo treatment of whole blood or leukocyte fractions. The method also facilitates evaluation of complementary and alternative medical treatments. In yet another embodiment, step (b) precedes or is concurrent with step (a).

Also provided herein are cells or monkeys infected with the recombinant human immunodeficiency virus 1 as described herein. Such cells or monkeys can be used for evaluating various potential therapeutic interventions, as mentioned above.

In a further embodiment, recombinant human viral vectors and methods are provided for evaluating gene therapy viral vectors in monkey models, and such vectors may be used without modification for human gene therapy as well. Often, gene therapy studies are hindered or risky because no suitable model exists to evaluate whether the target therapeutic gene in a HIV-1-based viral vector can be efficiently delivered and/or expressed in a species other than humans, and regulatory bodies favor conducting safety and efficacy studies in non-human species including primates, prior to human exposure. Viral vectors and in one non-limiting embodiment lentiviral-based gene therapy vectors, such as HIV-1-based viral vectors, may not deliver the therapeutic gene in monkeys or simian cells because of TRIM5α restriction of the human capsid, for the reasons described above. Thus, a different vector than that intended for human studies may need to be constructed to carry out prescribed safety and efficacy testing prior to human testing. Thus, in one embodiment, viral vectors are provided, wherein the viral vector overcomes simian cell capsid targeted restriction, such as TRIM restriction, thus permitting delivery of the vector genome and the target therapeutic gene into the simian cells as well as human cells. In one embodiment TRIM restriction is TRIM5α restriction. In another embodiment, the viral vector is capable of delivering the therapeutic gene into normal simian cells, transformed simian cells, normal human cells, transformed human cells, humans or monkeys. In a further embodiment, the simian cells are rhesus macaque cells, pigtail macaque cells or African green monkey cells, and the monkeys are rhesus macaques, pigtail macaques or African green monkeys. In another embodiment, TRIM5α restriction is overcome by replacement of the human immunodeficiency virus 1 capsid sequence or a fragment thereof by the simian immunodeficiency virus capsid sequence or a fragment thereof, such as wherein residues 1-202 of the simian immunodeficiency virus capsid sequence replace residues 1-204 of the human immunodeficiency virus 1 capsid sequence. In another embodiment, the simian immunodeficiency virus is MAC₂₃₉. In a further embodiment the virus comprises a functional envelope. In one embodiment, the envelope is from vesicular stomatitis virus. In a further embodiment, the virus comprises mutations in Gag selected from E12K, K110I, A208V, P371L, or any combination thereof, or a silent mutation in nucleotides 291, 321, or 477 in gag, or 579, 1248, 2149, 2157 or 3411 in pol, or any combination of any of the foregoing. In another embodiment, Gag K110I, A208V and P371L mutations are present.

In another embodiment, a method is provide for evaluating the effectiveness of therapeutic gene delivery using human viral vectors in monkeys comprising the steps of (a) exposing simian cells or monkeys to the recombinant viral vector comprising a therapeutic gene described above, (b) determining the level of expression of the therapeutic gene therein or the therapeutic effect thereon, and (c) correlating the expression or effect with the ability of the recombinant lentivirus to deliver the therapeutic gene. In one embodiment, the simian cells are T cells. In another embodiment, the simian cells are normal simian cells or transformed simian cells, and the simian cells are rhesus macaque cells, pigtail macaque cells or African green monkey cells. In another embodiment the monkey is a rhesus macaque, a pigtail macaque or an African green monkey.

In an example of the practice of this embodiment of the invention using HIV-1 components as the viral vector and eliminating simian cell restriction thereby, the HIV(SCA) Gag-pol region as described above was cloned into the pCRV1 vector. This vector is designed to express genes under the control of the CMV promoter and contains the following HIV-1 sequences: RU5 region and the non-coding sequence following the 5′LTR including the splice donor and acceptor sites and the coding sequences for Tat, Rev and Vpu. A multiple cloning site was introduced in the HIV-1 non-coding region. The Gag sequence was amplified from a HIV(SCA) proviral plasmid introducing an EcoRI site immediately 5′ to the Gag start codon and using a reverse primer at the end of the gag coding sequence. The PCR product was digested using EcoRI-ApaI (a site in the NC coding region) and cloned into a pCRV1 vector expressing the wild type HIV-1 Gag-pol, thus replacing the EcoRI-ApaI sequence of HIV-1 by that of HIV(SCA). The vector further comprises a functional envelope, such as may be from vesicular stomatitis virus. Such envelope provides infectivity to the virus but the envelope gene is not delivered to the target cells.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: kDa (kilodalton); rec. (recombinant); N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); ELISA (enzyme linked immunosorbent assay); mAb (monoclonal antibody); APC (antigen presenting cell); CTL (cytotoxic T lymphocyte); PBMC (peripheral blood mononuclear cells); Th (helper T); IFNγ (interferon-γ); HIV (human immunodeficiency virus); SIV (simian immunodeficiency virus), SHIV (simian human immunodeficiency virus); stHIV (simian tropic HIV), PCR (polymerase chain reaction); RT (reverse transcriptase).

### Example 1

### Production and In Vitro Characterization of Exemplary stHIV Virions and stHIV-Based Retroviral Gene Therapy Vectors

This example describes the production and characterization of recombinant HIV(SCA), HIV(SVif) and HIV(SCA,SVif) vectors and their use for the production of simian tropic HIV (stHIV) virions.

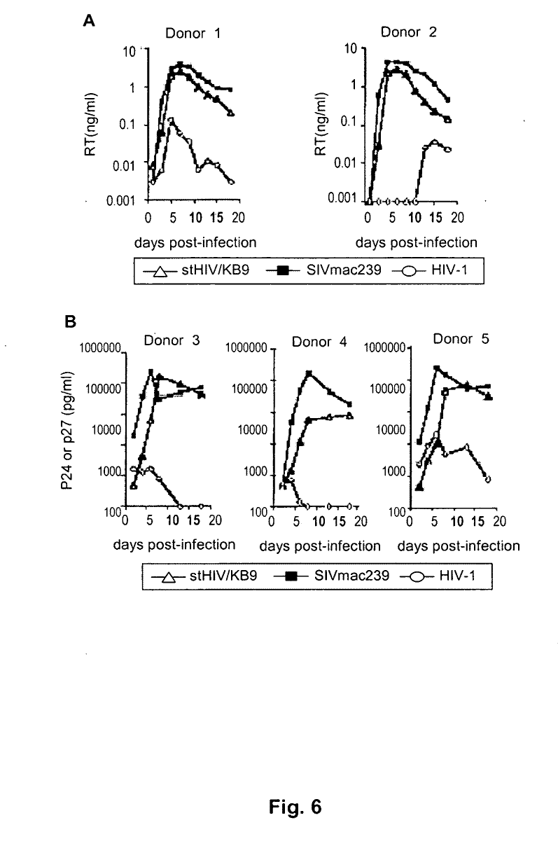

Cells/cell lines including various human and rhesus macaque cell lines as well as primary rhesus macaque PBMC and CD4+ enriched T-cells. 293T cells, TE671 cells, FRhK cells, GHOST cells (HOS cells expressing CD4, CXCR4 and a GFP reporter gene under the control of an HIV-2 LTR) and TZM cells (HeLa cells expressing CD4, CXCR4, CCR5 and a Luciferase reporter gene under the control of an HIV-1 LTR) were maintained in DMEM/10% fetal bovine serum/antibiotics supplemented with G418, puromycin and/or hygromycin where appropriate. The human T-cell line HuT/CCR5 and the human T-/B-hybrid cell line CEMx174 were maintained in RPMI/10% FCS/antibiotics. To generate CEMx174 cells stably expressing rhTRIM5, cells were inoculated with retroviral vector stocks generated by co-transfecting 293T cells with an LNCX2-based vector (Clontech) expressing rhTRIM5a and plasmids expressing MLV Gag-Pol and VSV-G. A single-cell clone derived from the resulting G418 resistant population, termed CEMxl74/rhTRIM5 was used in these studies. The Herpesvirus saimiri transformed rhesus monkey T cell line, 221(52) was maintained in RPMI/20% FCS/antibiotics supplemented with 60 units/ml IL-2. Rhesus macaque peripheral blood mononuclear cells (PBMC), used in FIG. 6B, were isolated by density gradient centrifugation and stimulated with 3 μg/ml Staphylococcus enterotoxin B and 20 U/ml IL-2 for three days, prior to washing and infection in the presence of IL-2. Alternatively For preparation of rhesus peripheral blood T lymphocytes enriched for CD4+ T cells (FIG. 6A), PBMCs were collected and washed twice with MACS buffer (PBS with 0.5% BSA), then pelleted. Residual erythrocytes were lysed by incubating with ACK lysis buffer for 5 min at 25° C. PBMC were then washed once in MACS buffer and incubated with anti-CD8 microbeads (Miltenyi Biosciences) in MACS buffer for 25 min at 4° C. with occasional agitation. CD8-positive cells were removed by negative selection on magnetic columns. The resulting CD8-depleted (CD4-enriched) fraction (typically >80% CD4+ cells by flow cytometric analysis) was collected and washed twice with cold MACS buffer. CD4-enriched cells were resuspended in RPMI 1640 (GIBCO) containing 10% fetal bovine serum, 1 mM glutamine, 100 units/ml penicillin and 100 μg/ml streptomycin (RPMI) and stimulated by plating 2-5×10⁶ PBMC/well overnight in 6 well plates containing plate bound anti-CD3 antibody (BD-Pharmingen). Cells were washed once with RPMI prior to infection.

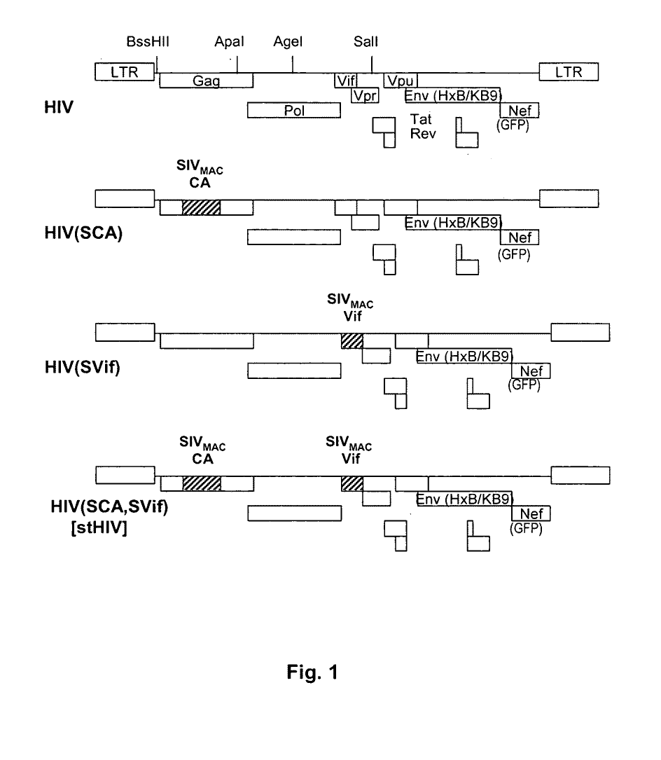

Proviral plasmid construction and nomenclature. Manipulations of the basic HIV-1 derived proviral genomes are depicted schematically in FIG. 1. White boxes represent HIV-1 open reading frames, shaded boxes represent SIVMAC239 counterparts. Env genes from two HIV-1 strains (HxB or KB9) were used, and some constructs encoded GFP in place of Nef. Restriction enzyme sites used for generating the various recombinant proviruses are indicated.

To generate an HIV-1 proviral plasmid clone encoding SIVMAC CA, two pairs of overlapping PCR primers were used in a recombinant PCR-based strategy. This approach employed primers that encoded sequences linking the C-terminus of HIV-1 matrix to the N-terminus of SIVMAC CA and residue 206 of the SIVMAC CA to residue 205 of HIV-1 CA. These primers were used in conjunction with primers spanning the BssHII site in the HIV-1 5′ untranslated region and the ApaI site in the HIV-1 nucleocapsid coding sequence in two sequential rounds of PCR. The recombinant PCR products were digested with BssHII and ApaI and inserted into an NL4-3-based proviral plasmid, termed HIV/HxB, containing env from HxB3 and an intact nef thereby generating HIV(SCA)/HxB. Genomic DNA was isolated from HIV(SCA)/HxB infected CEMx174 cells (Qiagen) following serial passage (see results section), and viral sequences were amplified by PCR using a 5′ primer spanning the BssHII restriction site in the 5′ untranslated HIV-1 region and a 3′ primer spanning the Gag termination codon. These PCR products were cloned and sequenced. The clone selected for further analysis was introduced into the HIV/HxB proviral plasmid using the BssHII and ApaI sites (FIG. 1) and named B3HIV(SCA)/HxB.

To generate HIV/KB9/GFP, the HxB envelope in an NL4-3-based proviral plasmid encoding the green fluorescent protein (GFP) in place of Nef (53) was replaced by the KB9 envelope (obtained from pSHIV-KB9) using KpnI and BamHI sites (FIG. 1). Subsequently, the BssHII-ApaI fragment of Gag (FIG. 1) was replaced by the corresponding sequence from B3HIV(SCA)/HxB to generate the B3HIV(SCA)/GFP/KB9 proviral plasmid.

To generate the HIV(SVif)/KB9/GFP proviral plasmid, the HIV-1 Vif encoding sequence in HIV/KB9/GFP was replaced by that encoding SIVMAC Vif. Specifically, HIV(SVif) was designed so that the 5′ end of the SIVMAC Vif coding sequence is positioned immediately 3′ to the HIV-1 Pol stop codon. Moreover, the 5′ end of the HIV-1 Vpr coding sequence is positioned immediately 3′ to the SIVMAC Vif stop codon. Additionally, several ATG codons found in the portion of the HIV-1 genome where HIV-1 Vif and Pol would normally overlap, as well as ATG codons normally found within the SIVMAC Vif were mutated, without changing the amino-acid coding potential of HIV-1 Pol or SIVMAC Vif respectively. The construct was made using recombinant PCR approaches. Specifically, two pairs of overlapping primers spanning (a) the 3′ end of HIV-1 Pol and the 5′ end of SIVMAC Vif and (b) the 3′ end of SIVMAC Vif and the 5′ end of HIV-1 Vpr were used in combination with a primers spanning either the AgeI site in HIV-1 Pol and a primer spanning the SalI site in HIV-1 Vpr (FIG. 1). The recombinant PCR product was initially introduced into the HIV/KB9/GFP proviral plasmid backbone using the Agel and SalI sites, thereby generating HIV(SVif)/KB9/GFP. Next, the BssHII-ApaI fragment of this plasmid was replaced by the corresponding sequence from B3HIV(SCA)/KB9/GFP to generate the B3HIV(SCA,SVif)/KB9/GFP proviral plasmid. Following serial passage of virus generated by B3HIV(SCA,SVif)/KB9/GFP in CEMxl74 and rhesus 221 cells (see results), genomic DNA was isolated from infected cells and a 5 kb DNA fragment comprising Gag, Pol and Vif encoding sequences was amplified by PCR using primers spanning the BssHII site in the 5′ untranslated region and the SalI site in Vpr. The Gag-Pol-Vif fragment (BssHII-SalI) from two clones was then transferred into the HIV/KB9 provirus (encoding and intact Nef protein and the KB9 envelope), thereby generating proviral clones of simian tropic HIV-1, stHIV/KB9.

Virus infectivity, replication and drug sensitivity assays. All virus stocks were generated by transfection of 293T cells with proviral plasmids. Viral yield and infectivity was quantified by RT assays (Cavidi Tech) and by titration in GHOST, CEMX174 or 221 cells, as indicated in the text. To measure the effect of rhAPOBEC3G on virion infectivity, proviral plasmids were cotransfected in 293T cells with a plasmid expressing rhAPOBEC3G(31) or a control at a 1:1 ratio. Single-cycle infectivity measurements were done using proviruses carrying GFP in place of Nef, or using reporter cell lines. Spreading replication was monitored using ELISA-based RT or CA detection assays. The sensitivity of viruses to antiretroviral drugs was determined as described previously (49, 51).

To measure single-cycle infectivity, GHOST cells (2×10⁴ per well in 48-well plates), CEMx174, CEMxl74/rhTRIM5α or 221 cells (1×10⁵ per well in 96-well plates) were inoculated with serial dilutions of each virus under test. After overnight incubation, dextran sulfate (100 μg/ml) was added to prevent secondary infections and cell fusion. Forty-eight hours post-infection the cells were harvested and analyzed using a Guava EasyCyte FACS analyzer.

Viral replication assays in CEM, 221 and unfractionated rhesus macaque PBMC were initiated by inoculating 5×10⁵ cells in 48 or 96-well plates with a volume of viral stock corresponding to 1 ng of RT. The day after infection cells were washed 3 times with medium and supernatant samples were harvested every 2 or 3 days thereafter.

Alternatively, for infection of enriched primary rhesus CD4+ T lymphocytes, cells were inoculated by placing 5×10⁵ cells in 0.2 ml RPMI, followed by addition of 0.5 ml of virus (corresponding to: 21 ng p27(CA) for ST-HIV(KB9); 136 ng p27(CA) for SIV_{MAC239}, and XX ng p24(CA) for HIV-1NL4-3 in 5 ml polypropylene snap-cap tubes. Cells were incubated with the virus inoculum for 2 hr at 37° C. after which an additional 0.8 ml RPMI was added to each tube and samples were incubated overnight at 37° C. Cells were then washed three times with RPMI and plated in individual wells of 24 well plates, with each well containing 5×10⁵ cells in 1.5 ml RPMI with 100 U/ml IL-2. Cultures were fed by removing 0.75 ml of culture medium every 48 hr and replacing with 0.75 ml fresh RPMI containing 200 U/ml IL-2. Harvested culture medium was reserved and assayed for virus production. Virus replication was monitored by assessment of supernatants using colorimetric ELISA-based RT assays (Cavidi Tech) or capsid antigen capture immunoassays for detection of HIV-1 p24 for HIV-1NL4-3 (Beckman Coulter) or SIV p27 (Zeptometrix) for SIV_{M}AC239 and stHIV/KB9.

The sensitivity of viruses to inhibition by RT inhibitors; (AZT; Sigma), nevirapine (NVP; AIDS Reference and Reagent Program) was determined as follows: TZM cells were inoculated, in duplicate, with HIV-1LAI, SIVMAC239, or stHIV/KB9 in the presence of 10 μg/ml of DEAE-dextran, with or without RT inhibitors. Cells were incubated for 2 hours at 37° C., washed with sterile PBS, received new medium with or without added inhibitor, and harvested 48 hours later. Luciferase activity in cell lysates was determined using the Luciferase Assay System (Promega) and a 1450 Microbeta luminescence counter (EG&G Wallac, Turku, Finland). Results were expressed as the percentage of the luciferase activity obtained for each virus in the absence of inhibitor.

Virus inhibition assays using the protease inhibitor amprenavir (APV, NIH AIDS Reference and Reagent Program) were performed with modifications as described previously (51). Briefly, HUT-R5 cells were infected with HIV-1LAI, SIVMAC239, or stHIV/KB9. When syncytia were visualized, the cells were washed and cultured overnight in the presence of increasing concentrations of inhibitor. Thereafter, culture supernatants were harvested and used to inoculate TZM-bl cells. Luciferase activity in cell lysates was determined as described above. Results were expressed as the percentage of the luciferase activity obtained for each virus in the absence of inhibitor.

To derive an HIV-1-based virus capable of replication in rhesus macaque cells, various modifications ware made to an HIV-1NL4-3 proviral plasmid clone. These proviral constructs (see FIG. 1), encoded the Env protein from the HxB3 strain or a rhesus macaque-adapted Env, termed KB9(34) and are referred to as HIV/HxB and HIV/KB9 respectively. Some constructs (e.g. HIV/HxB/GFP and HIV/KB9/GFP) encoded GFP in place of Nef. Additional modifications were made and are indicated in parentheses in the text. For example, ‘(SCA)’ and ‘(SVif)’ indicate that the CA and Vif portions of the genome, respectively, originate from SIV_{MAC239} (FIG. 1).

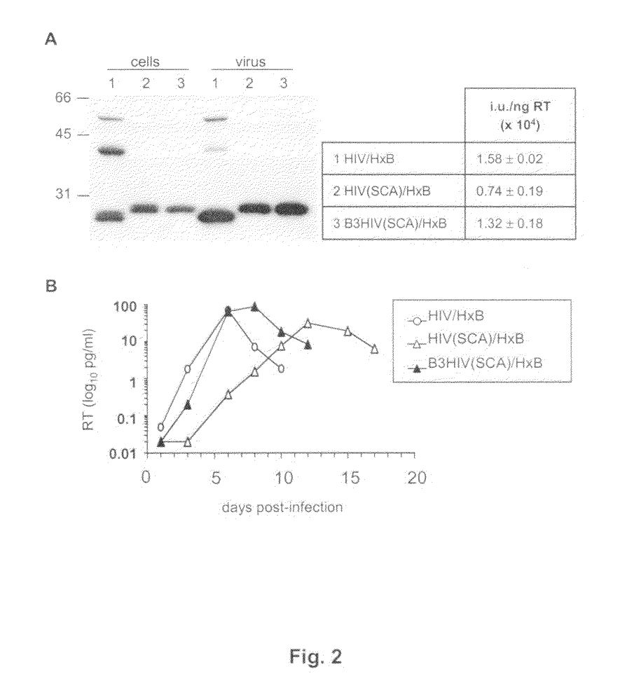

Adapting an HIV-1 strain encoding SIV_{MAC} CA. Previously it has been shown that the HIV-1 CA domain can be replaced with the SIV_{MAC} counterpart in the context of single-cycle infectious HIV-1 vectors. This manipulation confers resistance to rhTRIM5α-mediated restriction but such chimeric vectors exhibit significantly impaired single-cycle infectivity (6, 8, 12). Consistent with this finding, a full-length HIV-1 strain in which HIV-1 CA was replaced by SIV_{MAC} CA (HIV(SCA)/HxB, FIG. 1), was clearly attenuated as compared to HIV/HxB but was, nonetheless, capable of spreading replication in human CEMxl74 cells (FIG. 2). To improve its replication capacity, HIV(SCA)/HxB was serially passaged. After 16 weeks (19 cell-free passages) the passaged virus appeared to replicate more robustly and, unlike the starting virus, induced abundant syncytium formation within 3 days of inoculation of approximately 10⁶ CEMx174 cells with 1 ml of viral supernatant. DNA was isolated from the HIV(SCA)/HxB infected cells and four clones (termed B1-B4) of a fragment comprising matrix, CA and part of nucleocapsid were derived. Each of these was inserted into an Env-defective HIV-1 GFP-reporter virus (HIV/Env-/GFP). Single-cycle infectivity assays, using VSV-G pseudotyped virus, revealed that one proviral clone, termed B3HIV(SCA)/Env-/GFP, generated virions with 3-fold greater infectivity in human TE671 and rhesus FRhK target cells than did HIV(SCA)/Env-/GFP (data not shown). Thus, the B3 clone of the adapted HIV(SCA)/HXB had apparently acquired advantageous mutations and was selected for further analysis. Notably, B3HIV(SCA)/HxB contained only 3 amino acid changes (K110I, A208V and P371L) in Gag compared to the nonadapted HIV(SCA)/HxB parent.

A comparison of B3HIV(SCA)/HxB with the non-adapted HIV(SCA)/HxB parent and HIV/HxB (FIG. 2) revealed that Gag expression and particle production by 293T cells transfected with the non-adapted HIV(SCA)/HxB provirus were only marginally reduced as compared to cells transfected with HIV/HxB (FIG. 2A). Surprisingly, although the anti-HIV-1 CA antibody used for the immunoblots also recognizes SIVMAC CA, we observed only weak bands corresponding to processed Gag precursor (55 kD) and processing intermediates (41 kD) for HIV(SCA)/HxB and B3HIV(SCA)/HxB as compared to HIV/HxB. At present it is unclear whether this is due to processing differences between wild type HIV-1 and HIV(SCA) chimeras, or due to differences of the antibody reactivity for incompletely processed Gag proteins containing SIVMAC CA. Although CA expression by the parental HIV(SCA)/HxB and adapted B3HIV(SCA)/HxB proviral plasmids were similar, the latter, like HIV/HxB, generated virions that were slightly more infectious in single-cycle assays (FIG. 2A). Nonetheless, the differences in single-cycle infectivity between HIV/HxB, HIV(SCA)/HxB and B3HIV(SCA)/HxB were small (around 2-fold, FIG. 2A). However, the adapted B3HIV(SCA)/HxB virus clone replicated in CEMx 174 cells almost as efficiently as HIV/HxB, with both viruses reaching the peak of replication 5-7 days postinfection (FIG. 2B) while the non-adapted HIV(SCA)/HxB virus only achieved peak replication levels at 12 days postinfection (FIG. 2B). Thus, minor genetic, biochemical and single-cycle infectivity changes associated with adaptation of HIV(SCA)/HxB resulted in a virus with enhanced replication capacity.

Generation of rhTRIM5α and rhAPBOBEC3G resistant HIV-1. Using the adapted SIVMAC CA-encoding HIV-1 clone, B3HIV(SCA), as a foundation, we next sought to generate an HIV-1 strain that was resistant to both rhTRIM5α and rhAPOBEC3G. The entire HIV-1 Vif coding region was replaced by that of SIVMAC Vif to generate a proviral plasmid termed HIV(SVif). Initially, this was done both in the context of an HIV-1 proviral plasmid and in a counterpart, B3HIV(SCA), encoding SIVMAC CA. Both constructs encoded GFP in place of Nef (FIG. 1). Additionally, the proviral plasmids used in these and subsequent experiments encoded the KB9 HIV-1 envelope, which was selected because it can support efficient infection of primary rhesus macaque cells (34, 35).

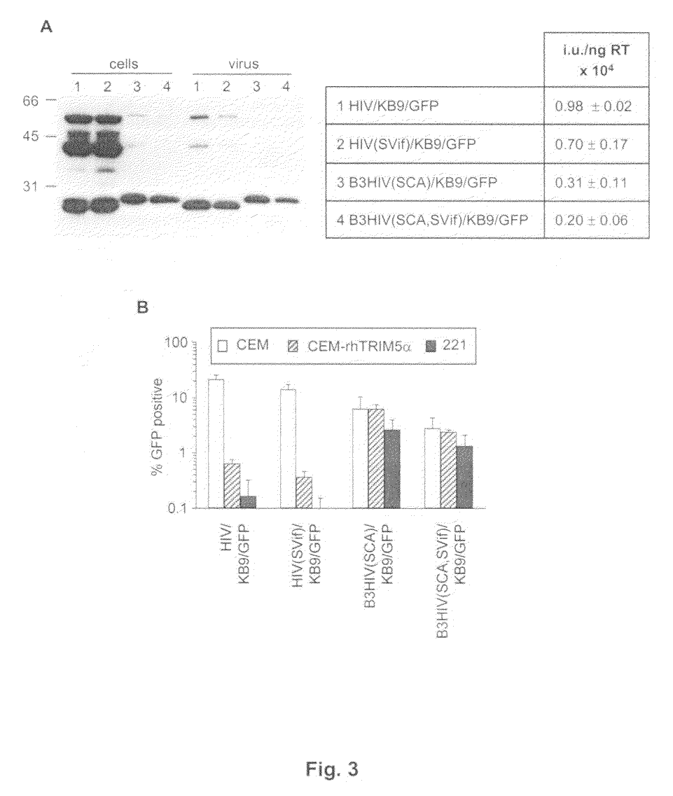

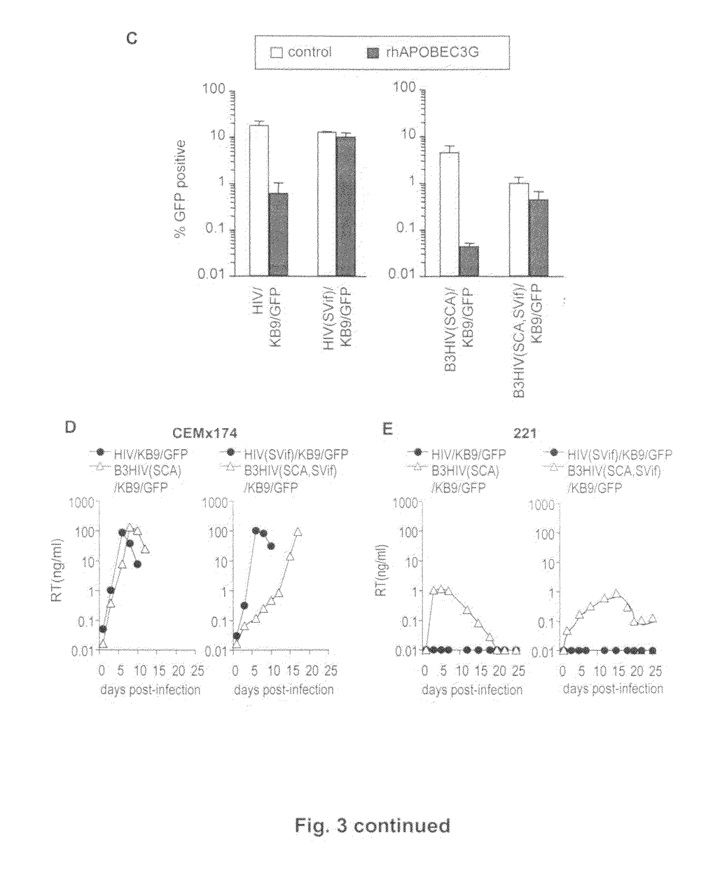

The proviral plasmids encoding the four possible combinations of HIV-1 and SIVMAC CA and Vif proteins (FIG. 1), namely HIV/KB9/GFP, B3HIV(SCA)/KB9/GFP, HIV(SVif)/KB9/GFP, B3HIV(SCA,SVif)/KB9/GFP were compared (FIG. 3). Surprisingly, Gag expression and particle production from 293T cell transfected with the B3HIV(SCA)-based proviral plasmids, was marginally reduced in this context, as compared to the HIV-1 CA-encoding equivalents, and single-cycle infectivity was also reduced approximately 3-fold (FIG. 3A). Nonetheless, the single-cycle infectivity of the B3HIV(SCA)-based viruses was similar in unmodified CEMx174 cells, in CEMx174 cells expressing rhTRIM5α and in rhesus macaque 221 cells. In contrast, infectivity of HIV-1 CA-encoding viruses was reduced 30-fold in TRIM5α-expressing CEMx174 and 70-fold in 221 cells as compared to unmodified CEMxl74 cells (FIG. 3B). Next, we determined whether the replacement of HIV-1 Vif by its SIVMAC counterpart in a proviral context conferred resistance to rhAPOBEC3G. The presence of rhAPOBEC3G during virus production reduced the single-cycle infectivity of HIV-1 Vif-encoding viruses by more than 30-fold (FIG. 3C). In contrast, rhAPOBEC3G had only marginal effects (less than 2-fold inhibition) on SIVMAC Vif-encoding counterparts (FIG. 3C). Overall, therefore, an HIV-1 clone engineered to express SIVMAC CA and Vif (B3HIV(SCA,SVif)/KB9/GFP, FIG. 3) was less infectious than HIV/KB9/GFP, but was resistant to both rhTRIM5α and rhAPOBEC3G.

Overcoming both rhTRIM5α and rhAPOBEC3G is required for HIV-1 replication in rhesus macaque cells. To determine whether the aforementioned modifications to HIV-1 were necessary and/or sufficient to confer tropism for rhesus macaque cells, the two pairs of viruses (FIG. 1) were compared in spreading infection assays in human CEMx 174 and rhesus 221 T-cell lines. Importantly, rhesus 221 cells are ‘non-permissive’ with respect to Vif function and do not support replication of Vif-defective SIVMAC(36).

Both HIV/KB9/GFP and B3HIV(SCA)/KB9/GFP replicated well in CEMx174 cells, reaching peaks of RT production at about day 6 and at day 8 post-infection, respectively (FIG. 3D). However, a comparison of HIV(SVif)/KB9/GFP with B3HIV(SCA,SVif)/KB9/GFP showed that the latter replicated more slowly, and supernatant RT activity peaked at day 17 post-infection, rather than day 6, though it eventually reached levels similar to those of HIV(SVif)/KB9/GFP (FIG. 3D).

As expected, HIV/KB9/GFP did not replicate in rhesus 221 cells (FIG. 3E). Moreover, while a low level of RT activity was generated by 221 cells inoculated with the B3HIV(SCA)/KB9/GFP virus during the first few days post-infection, levels decreased thereafter and no cytopathic effects were observed. Thus B3HIV(SCA)/KB9/GFP was capable of infecting rhesus macaque cells, but appeared unable to establish persistent replication (FIG. 3E). HIV(SVif)/KB9/GFP also failed to replicate in 221 cells, but infection with B3HIV(SCA,SVif)/KB9/GFP yielded progressively increasing levels of RT (FIG. 3E). Moreover, cytopathic effects including syncytium formation became evident at 16-20 days postinfection. Of note, the rhesus 221 cells (FIG. 3E) were challenged with 10-fold larger inocula than CEMx174 cells (FIG. 3D). Nevertheless, unlike any other viral clone, B3HIV(SCA,SVif)/KB9/GFP was clearly capable of replicating in 221 cells, demonstrating that overcoming both CA- and Vif-based host restriction is necessary and also sufficient for HIV-1 to establish productive infection in a rhesus macaque T-cell line.

Generation of a simian-tropic (st) HIV-1. Since B3HIV(SCA,SVif)/KB9/GFP replicated quite poorly, even in human CEMx174 cells, we sought to improve its replicative capacity by in vitro passage. This adaptation was done for 4 weeks (2 cell-free passages) in CEMx174 cells at which point cell-free passage into rhesus 221 cells was attempted. Within 14 days of inoculation, almost 100% of the 221 cells became GFP-positive and cytopathic effects were evident. Thereafter, cell-free supernatant readily reinitiated spreading infection in fresh 221 cells. After two further cell-free passages, DNA was isolated from the B3HIV(SCA,SVif)/KB9/GFP infected 221 cells and a 5 kb DNA fragment comprising Gag, Pol and Vif encoding sequences was amplified using PCR. Two clones were transferred into an intact proviral plasmid (HIV/KB9), thereby generating proviral clones of simian tropic HIV-1, stHIV/KB9.

Even following this second cycle of adaptation, stHIV/KB9 differed from the parental HIV/KB9 construct in only minor ways (other than the engineered substitution of CA and Vif proteins). Specifically, stHIV/KB9 clone1 had coding changes (amino acids K110I, A208V and P371L) as well as silent mutations in gag (nucleotides 291, 321 and 477) and pol (nucleotides 1248, 2157 and 3411). stHIV/KB9 clone 2 had coding changes (amino acids E12K, K110I, A208V and P371L) and silent mutations in gag (nucleotides 291, 321) andpol (nucleotides 579 and 2149). No changes in vif or the surrounding sequences were evident. The two clones of stHIV/KB9 behaved indistinguishably from each other and were used interchangeably in subsequent experiments.

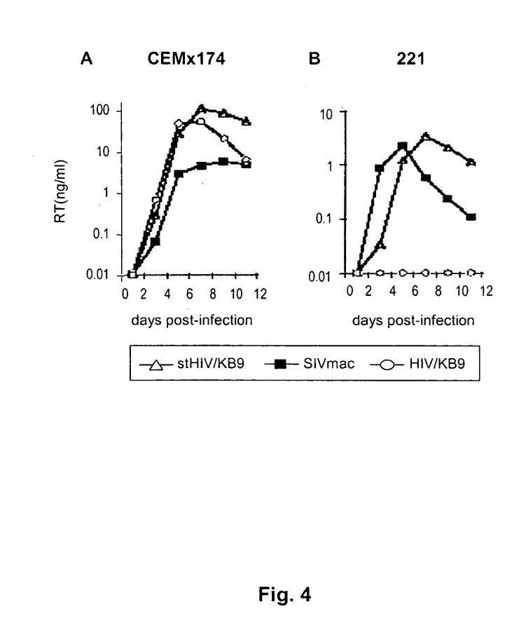

Spreading replication assays in human CEMx174 and rhesus macaque 221 cell lines revealed that HIV/KB9 and stHIV/KB9 replicated with similar kinetics in CEMx174 cells (FIG. 4A), indicating the latter had little or no intrinsic replication defect. SIVMAC239 replication kinetics in CEMx174 cells was also similar, although RT accumulated to a reduced level (FIG. 4A).

Importantly, stHIV replicated robustly in rhesus 221 cells, and achieved the same level of RT accumulation as did SIVMAC239, almost as rapidly. Indeed, stHIV replication peaked in rhesus macaque 221 cells at day 6-7 post-infection, as it did in human CEMx174 cells (FIG. 4B). In contrast, HIV/KB9 replication was undetectable in 221 cells. Thus, replacement of CA and Vif, combined with minimal additional changes associated with adaptation, enabled efficient HIV-1 replication in a rhesus macaque T-cell line.

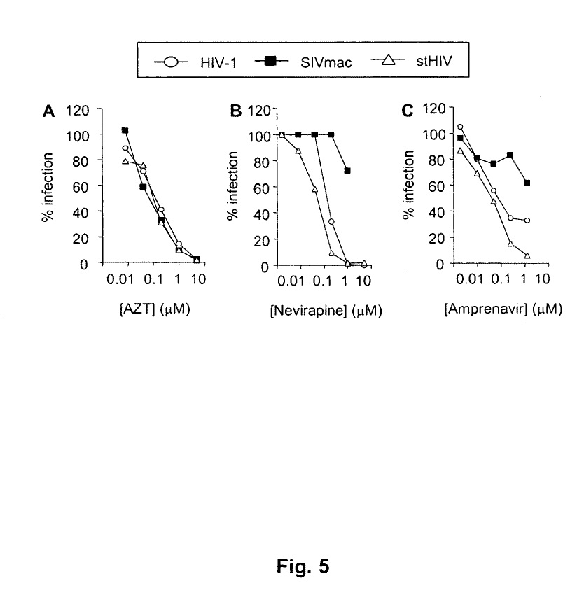

stHIV sensitivity to HIV-1-specific antiretroviral drugs. Because the bulk of the stHIV genome, including sequences encoding protease and RT, is of HIV-1 origin, stHIV was expected to retain sensitivity to HIV-1-specific antiretroviral agents. Two antiretroviral drugs that are highly effective for HIV-1 treatment and/or prophylaxis, but are ineffective in SIV/nonhuman primate models, are nevirapine, a nonnucleoside RT inhibitor, and amprenavir, a protease inhibitor. Both were effective inhibitors of HIV-1NL4-3 but ineffective or poorly inhibitory against SIV_{MAC239} (FIG. 5). Importantly, stHIV/KB9 behaved like HIV-1_{NL4-3} in terms of its sensitivity to nevirapine and amprenavir and was potently inhibited by both. As a control, the sensitivity of HIV-1, SIV_{MAC239} and stHIV/KB9 to AZT was tested and each virus was found to be equally sensitive (FIG. 5).

stHIV replicates robustly in primary rhesus macaque T-cells. While stHIV/KB9 replicated efficiently in the transformed rhesus macaque 221 T-cell line, it was possible that primary rhesus macaque cells might impose additional blocks to replication. Therefore, stHIV/KB9 replication was tested in primary rhesus macaque cells. First, stHIV/KB9 replication was compared to that of HIV-1_{NL4.3} and SIV_{MAC239} in enriched rhesus macaque CD4+ T-cells (FIG. 6A). As expected, HIV-1_{NL4-3} replication was undetectable in cells from two of the three macaque donors. While some replication was apparent in a third donor, levels of CA in culture supernatants only reached about 1% of the level achieved by SIVMAC239 (FIG. 6A). In contrast, stHIV/KB9 rapidly established high levels of replication in CD4+ T-cells from all three donors, and CA protein accumulated in culture supernatants to levels between 100 ng/ml and 1 μg/ml within 6 to 8 days of infection. In a separate experiment, unfractionated peripheral blood mononuclear cells (PBMC) from two macaque donors were inoculated with stHIV/KB9, HIV-1/KB9 or SIVMAC239 (FIG. 6B). In PBMC from both donors, SIVMAC239 and stHIV-1 replicated with similar rapid kinetics and to similar high levels, while HIV/KB9 replication was either extremely low (donor 5, FIG. 6B) or undetectable (donor 4). Thus stHIV/KB9 could replicate in primary rhesus macaque T-cells in vitro with an efficiency approaching that of SIVMAC239.

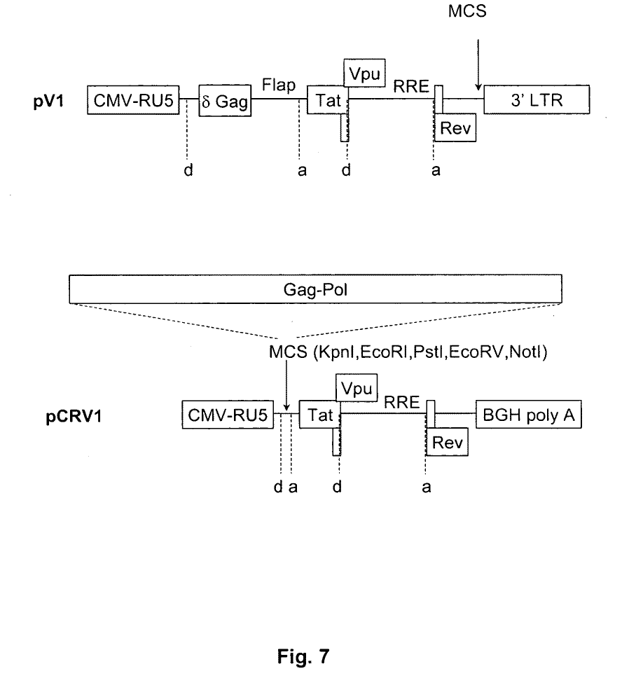

Viral vectors for human gene therapy capable of delivering therapeutic genes to simian cells and monkeys as well as humans. The HIV(SCA) Gag-pol region was cloned into the pCRV1 vector (see FIG. 7). This vector is designed to express genes under the control of the CMV promoter and contains the following HIV-1 sequences: RU5 region and the non-coding sequence following the 5′LTR including the splice donor and acceptor sites and the coding sequences for Tat, Rev and Vpu. A multiple cloning site was introduced in the HIV-1 non-coding region. The Gag sequence was amplified from a HIV(SCA) proviral plasmid introducing an EcoRI site immediately 5′ to the Gag start codon and using a reverse primer at the end of the gag coding sequence. The PCR product was digested using EcoRI-ApaI (a site in the NC coding region) and cloned into a pCRV1 vector expressing the wild type HIV-1 Gagpol, thus replacing the EcoRI-ApaI sequence of HIV-1 by that of HIV(SCA). The sequence of this Gagpol plasmid is identical to that of NL4.3 with amino acids 1-204 of capsid being replaced by amino acids 1-202 of SIVmac and contains the following adaptive changes: K110I, A208V and P371L.

### Example 2

### Production of stHIV Variants and Characterization in Rhesus Macaques

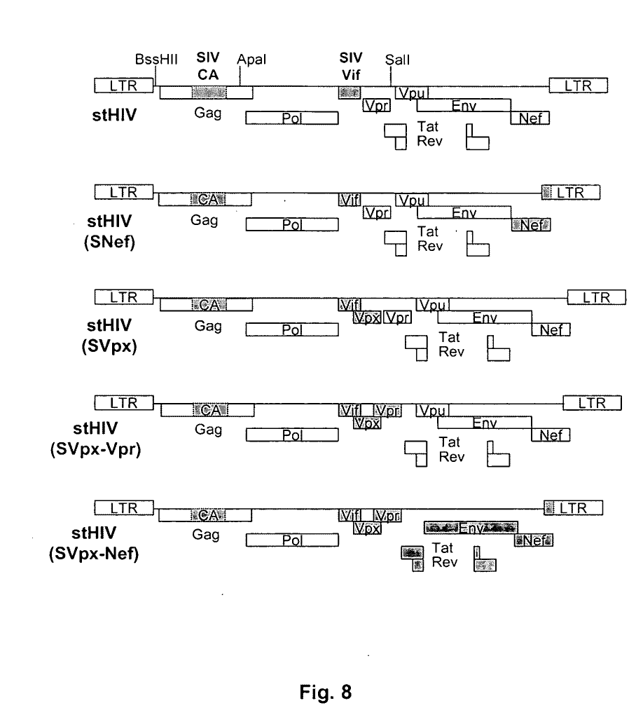

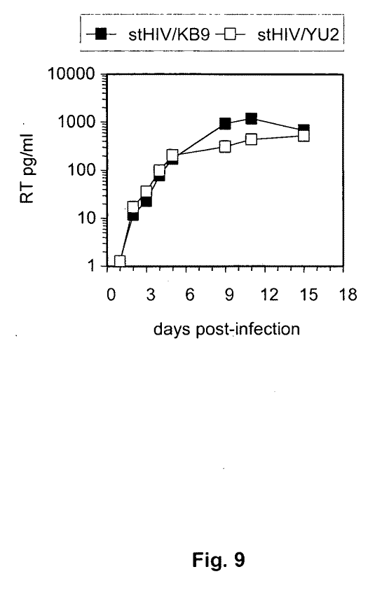

This example describes the production and characterization of recombinant stHIV(SVpx), stHIV(SVpx/Vpr), stHIV(SNef), and stHIV(SVpx-Nef) vectors and their use for the production of simian tropic HIV (stHIV) virions. Briefly HIV-1 plasmids comprising one or more SIV_{MAC} polynucleotides are produced. As shown in the schematic of FIG. 8, all of the stHIV variants of this example were engineered to express SIV_{MAC} capsid and Vif. The stHIV(SVpx) vector was produced by further engineering stHIV of Example 1, to express SIV_{MAC} Vpx. The stHIV(SVpx/Vpr) vector was produced by further engineering stHIV to express SIV_{MAC} Vpx and Vpr in place of HIV-1 Vpr. The stHIV(SNef) vector was produced by further engineering stHIV to express SIV_{MAC} Nef in place of HIV-1 Nef. Three versions of this plasmid exist, containing one, two or three NFkB sites prior to the TATA box in the 3′ HIV-1 LTR. The stHIV(SVpx-Nef) vector was produced by further engineering stHIV to express SIV_{MAC} Vpx, Vpr, Tat, Rev, Env and Nef. Unless noted otherwise, all vectors encode an HIV-1_{KB9} envelope. However, other HIV-1 envelopes can be used in the stHIV chimeras of the present invention, such as the HIV-1_{YU2} (FIG. 9).

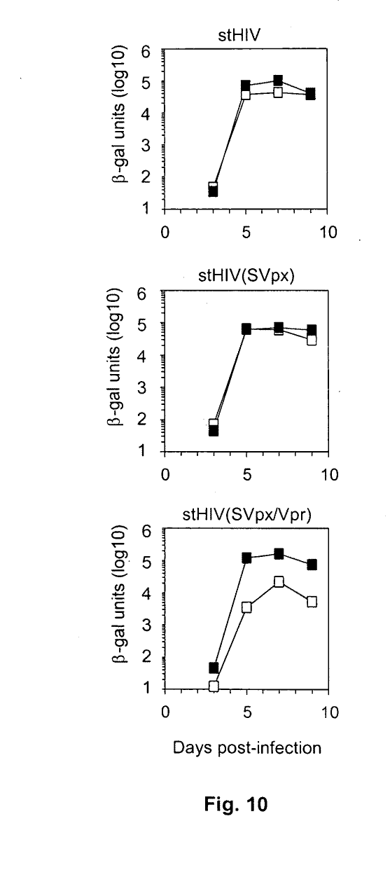

In vitro replication. For the viruses shown in FIG. 8, it is generally useful to optimize their replication capacity by serial in vitro passage. This has been done for stHIV and is being done for the stHIV variants. In the stHIV(SVpx) chimera, SIV_{MAC} Vif-Vpx coding sequences replace SIV_{MAC} Vif in the stHIV(SCA/SVif) vector. The HIV-1 Vpr coding sequence commences immediately 3′ to the SIV_{MAC} Vpx stop codon. In the stHIV(SVpx/Vpr) chimera SIV_{MAC} Vif, Vpx and Vpr coding sequences replace HIV-1 Vif and Vpr coding sequences up to the Tat start codon in the stHIV(SCA/SVif) vector. Gag expression, processing and infectious particle production in human 293T cells was confirmed for both chimeras. stHIV(SVpx) replicated in 221 cells as efficiently as stHIV, whereas stHIV(SVpx/Vpr) replication was somewhat delayed and the virus released from 221 cells at the peak of replication was not as infectious as stHIV. Therefore, the fitness of this chimera was improved by serial passage in 221 cells, as was done for stHIV. After 24 cell-free passages (pass24) the replicative capacity of the viruses harvested from 221 cells was compared to that of the original viral stock. As shown in FIG. 10, stHIV(SVpx/Vpr) (pass24) virus replication was improved significantly compared to the parental stocks.

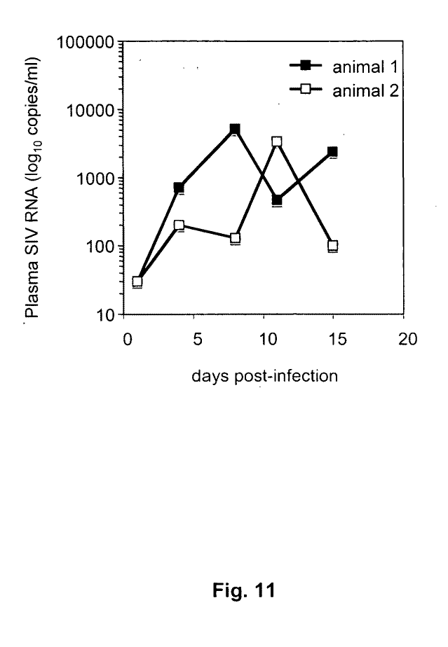

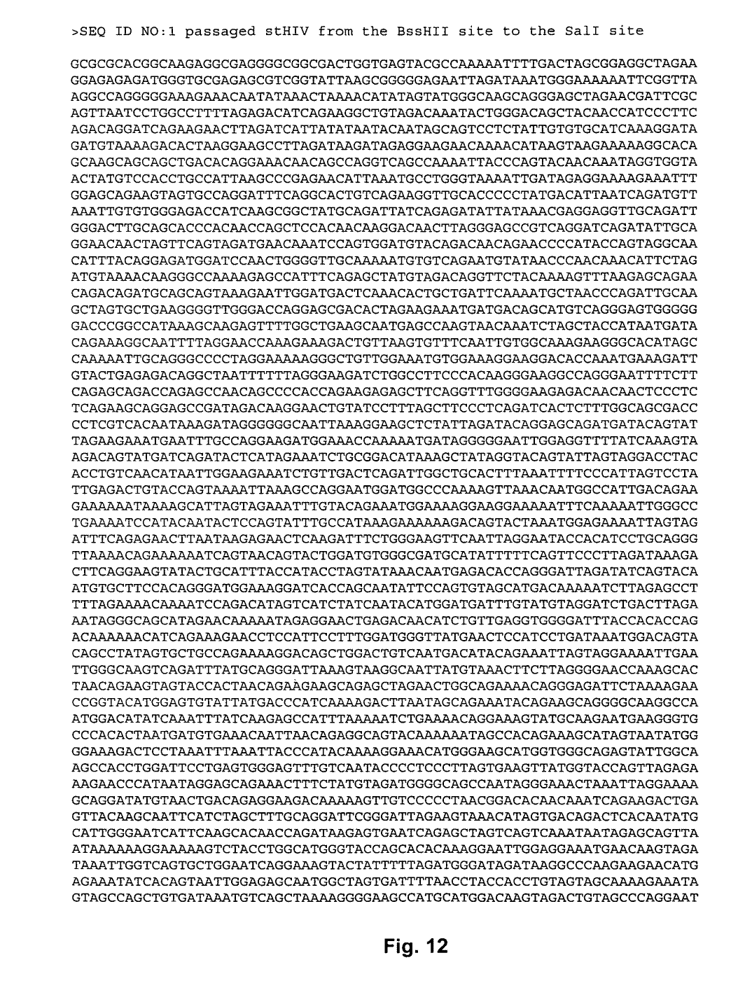

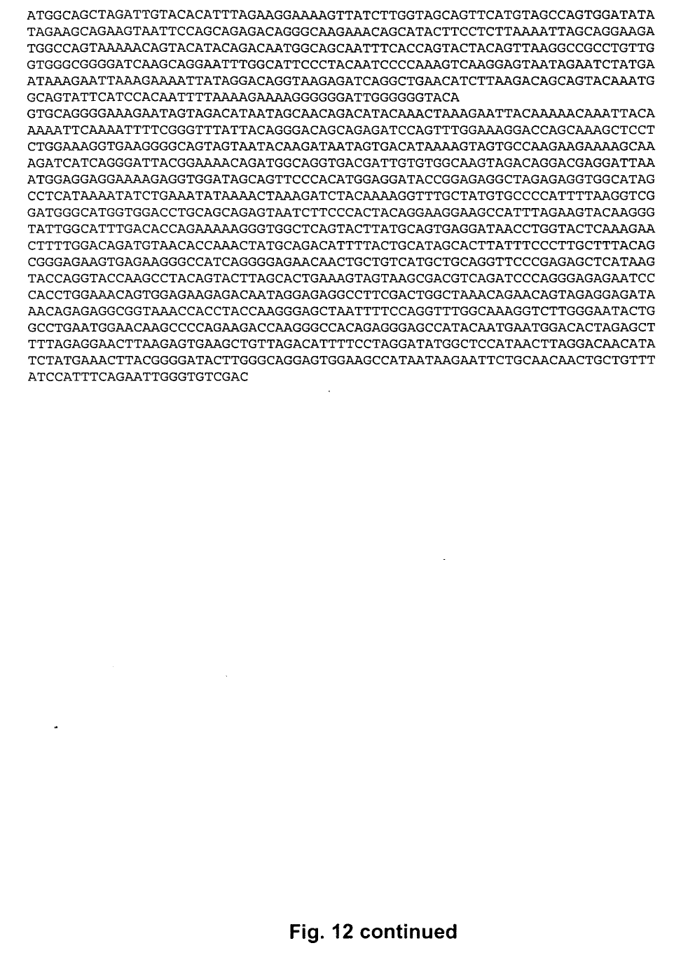

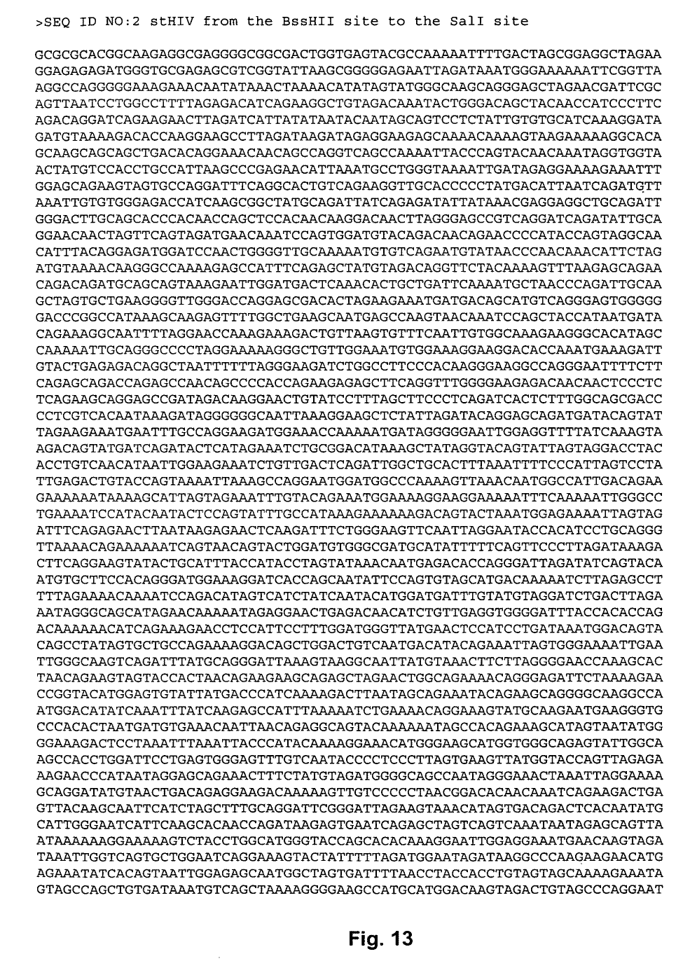

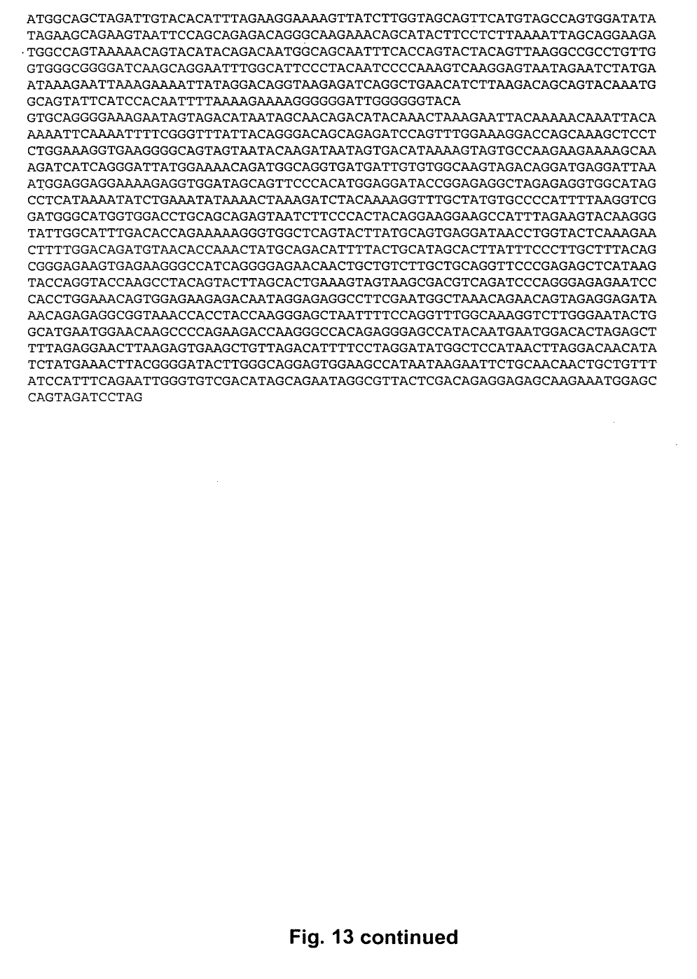

In vivo replication in rhesus macaques. The original stHIV (stHIV/KB9) has been tested in animals as shown in FIG. 11. Briefly, two rhesus macaques were inoculated with stHIV/KB9. Each animal was intravenously injected with 2 ml of supernatant with titers of 5×10⁵ i.u./ml (as measured on GHOST cells, an indicator cell line expressing GFP under the control of the HIV-1 LTR as well as CD4, X4 and R5 receptors). The two animals inoculated with stHIV/KB9 were monitored for CD4⁺ T-cell counts and viral load as known in the art. stHIV/KB9 replicated in both animals to modest levels, peaked at 10³-10⁴ RNA copies per ml but was eventually cleared. Although the levels of stHIV/KB9 replication are significantly lower than those generally observed with SIV_{MAC} isolates, it is remarkable that a measurable level of replication was observed for stHIV/KB9 in vivo in rhesus monkeys. Of note, SHIV chimeras that express a single HIV-1 protein in an otherwise SIV_{MAC} background required extensive in vivo adaptation to achieve high levels of replication and pathogenicity in vivo. Thus, improvements to the replication efficiency of stHIV candidates are contemplated to be achievable by serial in vivo passage.

### LITERATURE CITED

1. Gardner and Luciw, (1989) Faseb J 3, 2593-606.
2. Gartner et al., (1994) J Med Primatol 23, 155-63.
3. Hofmann et al., (1999) J Virol 73, 10020-8.
4. Cowan et al., (2002) Proc Natl Acad Sci USA 99, 11914-9.
5. Hatziioannou et al., (2003) Embo J 22, 385-94.
6. Owens et al., (2003) J Virol 77, 726-31.
7. Besnier et al., (2002) Proc Natl Acad Sci USA 99, 11920-5.
8. Stremlau et al., (2004) Nature 427, 848-53.
9. Munk et al., (2002) Proc Natl Acad Sci USA 99, 13843-8.
10. Kootstra et al., (2003) Proc Natl Acad Sci USA 100, 1298-303.
11. Perez-Caballero et al., (2005) J Virol 79, 15567-72.
12. Hatziioannou et al., (2004) Proc Natl Acad Sci USA 101, 10774-9.
13. Keckesova et al., (2004) Proc Natl Acad Sci USA 101, 10780-5.
14. Yap et al., (2004) Proc Natl Acad Sci USA 101, 10786-91.
15. Perron et al., (2004) Proc Natl Acad Sci USA 101, 11827-32.
16. Bieniasz, (2004) Nat Immunol 5, 1109-15.
17. Goff, (1996) Cell 86, 691-3.
18. Sheehy et al., (2002) Nature 418, 646-50.
19. Mehle et al., (2004) J Biol Chem 279, 7792-8.
20. Sheehy et al., (2003) Nat Med 9, 1404-7.
21. Yu et al., (2003) Science 302, 1056-60.
22. Harris and Liddament, (2004) Nat Rev Immunol 4, 868-77.
23. Marin et al., (2003) Nat Med 9, 1398-403.
24. Harris et al., (2003) Cell 113, 803-9.
25. Mangeat et al., (2003) Nature 424, 99-103.
26. Zhang et al., (2003) Nature 424, 94-8.
27. Lecossier et al., (2003) Science 300, 1112.
28. Bishop et al., (2004) Curr Biol 14, 1392-6.
29. Wiegand et al., (2004) Embo J 23, 2451-8.
30. Simon et al., (2005) PLoS Pathog 1, e6
31. Zennou and Bieniasz, (2006) Virology 349, 31-40.
32. Liddament et al., (2004) Curr Biol 14, 1385-91.
33. Mariani et al., (2003) Cell 114, 21-31.
34. Karlsson et al., (1997) J Virol 71, 4218-25.
35. Reimann et al., (1996) J Virol 70, 6922-8.
36. Kar et al., (2003) J Gen Virol 84, 3227-31.
37. Pekrun et al., (2002) J Virol 76, 2924-35.
38. Shibata et al., (1991) J Virol 65, 3514-20.
39. Li et al., (1992) J Acquir Immune Defic Syndr 5, 639-46.
40. Luciw et al., (1995) Proc Natl Acad Sci USA 92, 7490-4.
41. Joag et al., (1996) J Virol 70, 3189-97.
42. Akiyama et al., (2003) J Gen Virol 84, 1663-9.
43. Harouse et al., (1999) Science 284, 816-9.
44. Nishimura et al., (2004) Proc Natl Acad Sci USA 101, 12324-9.
45. Desrosiers, (2004) Nat Med 10, 221-3.
46. Witvrouw et al., (2004) Antivir Ther 9, 57-65.
47. Wyand, (1992) AIDS Res Hum Retroviruses 8, 349-56.
48. Boyer et al., (2006) PLoS Pathog 2, e10.
49. Ambrose et al., (2004) J Virol 78, 13553-61.
50. Uberla et al., (1995) Proc Natl Acad Sci USA 92, 8210-4.
51. Vaillancourt et al., (1999) AIDS Res Hum Retroviruses 15, 355-63.
52. Alexander et al., (1997) J Virol 71, 6094-9.
53. Zhang et al., (2002) J Virol 76, 6332-6343.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields, are intended to be within the scope of the following claims.

What is claimed is:

1. A proviral vector comprising:
i) nucleotides encoding a macaque simian immunodeficiency virus (SIVmac) capsid protein or fragment thereof in place of nucleotides encoding a human immunodeficiency virus 1 (HIV-1) capsid protein or fragment thereof;
ii) nucleotides encoding a SIVmac Vif protein in place of nucleotides encoding a HIV-1 Vif protein; and
iii) a sufficient number of nucleotides of an HIV-1 genome to provide a chimeric immunodeficiency virus genome suitable for use in producing a chimeric immunodeficiency virus upon introduction into permissive human cells or permissive nonhuman primate cells.

2. The proviral vector of claim 1, comprising nucleotides encoding an HIV-1 Vpr protein.

3. The proviral vector of claim 2, comprising nucleotides encoding a SIVmac Vpx protein.

4. The proviral vector of claim 1, comprising nucleotides encoding both a SIVmac Vpx protein and a SIVmac Vpr protein in place of nucleotides encoding an HIV-1 Vpr protein.

5. The proviral vector of claim 1, comprising nucleotides encoding a SIVmac Nef protein in place of nucleotides encoding an HIV-1 Nef protein.

6. The proviral vector of claim 1, comprising nucleotides encoding a SIVmac Vpx protein, a SIVmac Vpr protein, a SIVmac Tat protein, a SIVmac Rev protein, a SIVmac Env protein, and a SIVmac Nef protein, in place of nucleotides encoding an HIV-1 Vpr protein, an HIV-1 Tat protein, an HIV-1 Rev protein, an HIV-1 Env protein, and an HIV-1 Nef protein.

7. The proviral vector of claim 1, comprising nucleotides encoding a green fluorescent protein in place of nucleotides encoding an HIV-1 Nef protein.

8. A chimeric immunodeficiency virus produced upon introduction of the proviral vector of claim 1, into permissive human cells or permissive nonhuman primate cells.

9. The chimeric immunodeficiency virus of claim 8, wherein said permissive nonhuman primate cells are selected from the group consisting of rhesus macaque cells, pigtail macaque cells, cynomolgus macaque cells, and African green monkey cells.

10. The chimeric immunodeficiency virus of claim 9, wherein said permissive nonhuman primate cells are within a monkey selected from the group consisting of rhesus macaque, pigtail macaque, cynomolgus macaque, and African green monkey.

11. A method for identifying a test agent that modulates human immunodeficiency virus 1 (HIV-1) infection in vitro comprising the steps of:
a) exposing nonhuman primate cells to either the proviral vector of claim 1, or to a chimeric immunodeficiency produced upon introduction of said proviral vector into permissive human cells or permissive nonhuman primate cells, to yield infected cells;
b) culturing said infected cells in the presence or absence of a test agent; and
c) measuring a correlate of infection of said cultured infected cells, whereby a difference in said correlate in the presence of said test agent as compared to in the absence of said test agent, indicates that said agent modulates HIV-1 infection in vitro.

12. The method of claim 11, wherein said nonhuman primate cells are selected from the group consisting of rhesus macaque cells, pigtail macaque cells, cynomolgus macaque cells, and African green monkey cells.

13. The method of claim 11, wherein said nonhuman primate cells are selected from the group consisting of peripheral blood mononuclear cells (PBMC), immortalized T cell lines, and immortalized monocyte cell lines.

14. The method of claim 11, wherein said correlate of infection is selected from the group consisting of viral growth, cell survival, reverse transcriptase activity, integrase activity, and protease activity.

15. A method for identifying a test agent that modulates human immunodeficiency virus 1 (HIV-1) infection in vivo comprising the steps of:
a) exposing a monkey to either the proviral vector of claim 1, or to a chimeric immunodeficiency virus produced upon introduction of said proviral vector into permissive human cells or permissive nonhuman primate cells, to yield an infected monkey;
b) treating said monkey with a test agent; and
c) measuring a correlate of infection of said treated infected monkey, whereby a difference in said correlate in said treated infected monkey as compared to an untreated infected monkey, indicates that said agent modulates HIV-1 infection in vivo.

16. The method of claim 15, wherein said monkey is selected from the group consisting of rhesus macaque, pigtail macaque, cynomolgus macaque, and African green monkey.

17. The method of claim 15, wherein said correlate of infection is selected from the group consisting of viral growth, CD4-positive cell depletion, AIDS symptoms, reverse transcriptase activity, integrase activity, protease activity, viral load, and immune response.

18. The method of claim 15, wherein said test agent is selected from the group consisting of a reverse transcriptase inhibitor, an integrase inhibitor, a protease inhibitor, an antibody and a vaccine.

19. A nonhuman primate cell infected with the chimeric immunodeficiency virus of claim 8.

20. A monkey infected with the chimeric immunodeficiency virus of claim 8.


SEQUENCE LISTING (2 sequences)

SEQ ID NO: 1 - DNA, 5257 bp, simian-tropic human immunodeficiency virus
gcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacactaaggaagccttagataagatagaggaagaacaaaacataagtaagaaaaaggcacagcaagcagcagctgacacaggaaacaacagccaggtcagccaaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggttgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggttgggaccaggagcgacactagaagaaatgatgacagcatgtcagggagtggggggacccggccataaagcaagagttttggctgaagcaatgagccaagtaacaaatctagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtaggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtgggcagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagctagtcagtcaaataatagagcagttaataaaaaaggaaaaagtctacctggcatgggtaccagcacacaaaggaattggaggaaatgaacaagtagataaattggtcagtgctggaatcaggaaagtactatttttagatgggatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattacggaaaacagatggcaggtgacgattgtgtggcaagtagacaggacgaggattaaatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcatgctgcaggttcccgagagctcataagtaccaggtaccaagcctacagtacttagcactgaaagtagtaagcgacgtcagatcccagggagagaatcccacctggaaacagtggagaagagacaataggagaggccttcgactggctaaacagaacagtagaggagataaacagagaggcggtaaaccacctaccaagggagctaattttccaggtttggcaaaggtcttgggaatactggcctgaatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgac

SEQ ID NO: 2 - DNA, 5316 bp, simian-tropic human immunodeficiency virus
gcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggttgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtgggcagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagctagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaaatggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgacgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggcatgaatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctag